(19) United States
(12) Patent Application Publication
(10) Pub. No.: US 2023/0355791 A1
(43) Pub. Date: Nov. 9, 2023

(54) GLYCAN-CONJUGATED ANTIBODIES BINDING TO FC-GAMMA RECEPTOR

(71) Applicant: Synaffix B.V., Oss (NL)

(72) Inventors: Floris Louis VAN DELFT, Nijmegen (NL); Sander Sebastiaan VAN BERKEL, Oss (NL); Remon VAN GEEL, Lithoijen (NL); Lianne LELIEVELDT, Nijmegen (NL)

(21) Appl. No.: 18/213,151

(22) Filed: Jun. 22, 2023

Related U.S. Application Data
(63) Continuation of application No. PCT/EP2021/087648, filed on Dec. 24, 2021.

(30) Foreign Application Priority Data
Dec. 24, 2020 (EP) 20217241.7

(51) Int. Cl. A61K 47/68

(52) U.S. Cl. CPC A61K 47/6811

(57) ABSTRACT

The present invention provides antibody-conjugates which are conjugated via the glycan and still bind to a cell comprising an Fc-gamma receptor. The antibody conjugates according to the invention have structure (1):

Ab-[(GlcNAc(Fuc)_{b}-(G)ₑ-(Su-(Z-L-(D)ᵣ)ₓ)ₛ]_{y} (1)

Herein, Ab is an antibody; GlcNAc is an N-acetylglucosamine moiety; Fuc is a fucose moiety; b is 0 or 1; G is a monosaccharide; e is an integer in the range of 4-10; Su is a monosaccharide; Z is a connecting group obtained by a cycloaddition or a nucleophilic reaction; L is a linker; D is a payload; s is 1 or 2; r is an integer in the range of 1-4; x is 1 or 2; y is 2 or 4.

### CROSS-REFERENCE TO RELATED APPLICATIONS

This application is a continuation of International Patent Application No. PCT/EP2021/087648 filed Dec. 24, 2021, which application claims priority to European Patent Application No. 20217241.7 filed Dec. 24, 2020, the contents of which are all incorporated herein by reference in their entireties.

### FIELD OF THE INVENTION

The present invention relates to the field of antibody-drug conjugates, in particular to antibody-drug conjugates obtained by conjugation of a payload through the antibody glycan, which retain binding to Fc-gamma receptors (FcγRs). The antibody-drug conjugates of the invention are for example suitable for the treatment of cancer.

### BACKGROUND OF THE INVENTION

Antibody-drug conjugates (ADC), considered as magic bullets in therapy, are comprised of an antibody to which is attached a pharmaceutical agent. The antibodies (also known as ligands) are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated payload to the tumour, via binding, internalization, intracellular processing and finally release of active catabolite. The payload may be a small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases. Finally, attachment of an oligonucleotide to an antibody selectively taken up by muscle cells is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

In the field of ADCs, a chemical linker is typically employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment). The cleavable linker may also contain a self-immolative unit, for example based on a para-aminobenzyl alcohol group and derivatives thereof. A linker may also contain an additional, non-functional element, often referred to as spacer or stretcher unit, to connect the linker with a reactive group for reaction with the antibody.

Currently, cytotoxic payloads include for example microtubule-disrupting agents [e.g. auristatins such as monomethyl auristatin E (MMAE) and monomethyl auristatin F (MMAF), maytansinoids, such as DM1 and DM4, tubulysins], DNA-damaging agents [e.g., calicheamicin, pyrrolobenzodiazepine (PBD) dimers, indolinobenzodiapine dimers, duocarmycins, anthracyclines], topoisomerase inhibitors [e.g. DXd, SN-38] or RNA polymerase II inhibitors [e.g. amanitin]. ADCs that have reached market approval include for example payloads MMAE, MMAF, DM1, calicheamicin, SN-38 and DXd, while various pivotal trials are running for ADCs based on duocarmycin, DM4 and PBD dimer. A larger variety of payloads is still under clinical evaluation or has been in clinical trials in the past, e.g. eribulin, indolinobenzodiazepine dimer, PNU-159,682, hemi-asterlin, doxorubicin, vinca alkaloids and others. Finally, various ADCs in late-stage preclinical stage are conjugated to novel payloads for example amanitin, KSP inhibitors, MMAD, and others.

With the exception of sacituzumab govetican (Trodelvy®), all of the clinical and marketed ADCs contain cytotoxic drugs that are not suitable as stand-alone drug. Trodelvy® is the exception because it features SN-38 as cytotoxic payload, which is also the active catabolite of irinotecan (an SN-38 prodrug). Several other payloads now used in clinical ADCs have been initially evaluated for chemotherapy as free drug, for example calicheamicin, PBD dimers and eribulin. but have failed because the extremely high potency of the cytotoxin (picomolar-low nanomolar IC₅₀ values) versus the typically low micromolar potency of standard chemotherapy drugs, such as paclitaxel and doxorubicin.

Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948, incorporated by reference, and for example studied by Li et al, Cancer Res. 2016, 76, 2710-2719, incorporated by reference. Generally spoken, cytotoxic payloads that are neutral will show bystander killing whereas ionic (charged) payloads do not, as a consequence of the fact that ionic species do not readily pass a cellular membrane by passive diffusion. Payloads with established bystander effect are for example MMAE and DXd. Examples of payloads that do not show bystander killing are MMAF or the active catabolite of Kadcyla (lysine-MCC-DM1).

ADCs are prepared by chemical attachment of a reactive linker-drug to a protein, a process known as bioconjugation. Many technologies are known for bioconjugation, as summarized in G. T. Hermanson, “Bioconjugate Techniques”, Elsevier, 3^{rd} Ed. 2013, incorporated by reference. Two main technologies can be recognized for random conjugation to antibodies, either based on acylation of lysine side chain or based on alkylation of cysteine side chain. Acylation of the ε-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference.

A frequent method for attachment of linker-drugs to azido-modified proteins is strain-promoted alkyne-azide cycloaddition (SPAAC). In a SPAAC reaction, the linker-drug is functionalized with a cyclic alkyne and the cycloaddition with azido-modified antibody is driven by relief of ring-strain. Conversely, the linker-drug is functionalized with azide and the antibody with cyclic alkyne. Various strained alkynes suitable for metal-free click chemistry are indicated in FIG. 1. Besides cyclooctyne, certain cycloheptynes are also suitable for metal-free click chemistry, as reported by Weterings et al., Chem. Sci. 2020, doi: 10.1039/d0sc03477k, incorporated by reference. Smaller strained alkynes may also be employed, however in most cases require in situ generation of the strained alkyne due to inherent instability.

Reaction of strained alkynes with tetrazine is also a metal-free click reaction. Moreover, tetrazines also react with strained alkenes (tetrazine ligation). Both strained alkynes and strained alkenes react with tetrazines via inverse electron-demand Diels-Alder (IEDDA) reactions, exhibiting remarkably fast kinetics. For example, reaction of trans-cyclooctene (TCO) with tetrazine is unrivalled in its reaction speed and such rapid reaction has enabled applications in rodent models and other large organisms, settings where only minimal reaction times and reagent concentrations are tolerated. Triazine and other heteroaromatic moieties can also undergo reaction with strained alkynes or alkenes. Notably, strained alkenes typically do not undergo reaction with azides. Various strained alkenes suitable for metal-free click chemistry are indicated in FIG. 2.

Besides azides, strained alkynes can also undergo reaction with a range of other functional groups, such as nitrile oxide, nitrone, ortho-quinone, dioxothiophene and sydnone. A list of couples of functional groups F and Q (=strained alkyne or strained alkene) for metal-free click chemistry is provided in FIG. 3. A comprehensive overview of metal-free click chemistries for bioconjugation, extending also beyond proteins (e.g. glycans, nucleic acids), is provided by Nguyen and Prescher, Nature rev. 2020, doi: 10.1038/s41570-020-0205-0, incorporated by reference.

Based on the above, a general method for the preparation of a protein conjugate, exemplified for a monoclonal antibody in FIG. 4, entails the reaction of a protein containing x number of reactive moieties F with a linker-drug construct containing a single molecule Q.

It has been shown by van Geel et al., Bioconj. Chem. 2015, 26, 2233-2242 and Verkade et al., Antibodies 2018, 7, 12, all incorporated by reference, that enzymatic remodelling of the native antibody glycan at N297 also enables introduction of an azide into the antibody by means of trimming with an endoglycosidase, then transfer of an azidosugar, suitable for attachment of cytotoxic payload using click chemistry (see FIG. 5). Chemical approaches have also been developed for site-specific modification of antibodies without prior genetic modification, as for example highlighted by Yamada and Ito, ChemBioChem. 2019, 20, 2729-2737.

A common strategy in the field of ADCs employs nihilation or removal of binding capacity of the antibody to Fc-gamma receptors (FcγRs), which has multiple pharmaceutical implications.

The first consequence of removal of binding to Fc-gamma receptors is the reduction of Fc-gamma receptor-mediated uptake of antibodies by e.g. macrophages or megakaryocytes, which may lead to dose-limiting toxicity as for example reported for Kadcyla® (trastuzumab-DM1) and LOP628. Selective deglycosylation of antibodies in vivo affords opportunities to treat patients with antibody-mediated autoimmunity. Removal of high-mannose glycoform in a recombinant therapeutic glycoprotein may be beneficial, since high-mannose glycoforms are known to compromise therapeutic efficacy by aspecific uptake by endogenous mannose receptors and leading to rapid clearance, as for example described by Gorovits and Krinos-Fiorotti, Cancer Immunol. Immunother. 2013, 62, 217-223 and Goetze et al, Glycobiology 2011, 21, 949-959 (both incorporated by reference). In addition, Van de Bovenkamp et al, J. Immunol. 2016, 196, 1435-1441 (incorporated by reference) describe how high mannose glycans can influence immunity. It was described by Reusch and Tejada, Glycobiology 2015, 25, 1325-1334 (incorporated by reference), that inappropriate glycosylation in monoclonal antibodies could contribute to ineffective production from expressed Ig genes.

Fc-gamma receptors can be divided into high affinity receptors (FcγRI, also known as CD64) and low affinity receptors (FcγRII, also known as CD32 and FcγRIII, also known as CD16). A Fc-gamma receptor can be activating (denoted with A, e.g. FcγRIIIA or CD16A). Fc-gamma receptors may be present at various expression levels on a variety of immune cells, including macrophages, monocytes, dendritic cells, neutrophils, NK cells and B cells (see FIG. 6), as summarized by Rosales, Front Immunol. 2017, 20, doi.org/10.3389/fimmu.2017.00280, and Castro-Dopico and Clatworthy, Curr. Transpl. Rep. 2016, 3, 284-293, both incorporated by reference.

Binding of an antibody to a specific Fc-gamma receptor is highly dependent on the IgG type. For example, IgG1 and IgG2 will bind to Fc-gamma receptor III, while IgG4 shows no or negligible binding. Also, the presence of the N-glycan in the antibody Fc-fragment strongly influences binding, with non-glycosylated antibodies showing no binding to low affinity receptors and significantly reduced binding to high affinity receptors, as for example reported by Lux et al., J. Immunol. 2013, 190, 4315-4323, incorporated by reference. Also, the specific nature of the N-glycan will heavily influence the binding affinity to various receptors, as for example reported by Wada et al., mAbs 2019, 11, 350-372, incorporated by reference.

The specific glycosylation profile of a monoclonal antibody can be directed by performing the recombinant expression in the presence of specific glycosidase inhibitors or glycosyl transferase inhibitors. For example, expression of an antibody in CHO or HEK293 expression platform in the presence of kifunensin will lead to inhibition of α-mannosidase I, thereby generating only high mannose form of the antibody, as for example reported by Zhou et al, Biotechnol. Bioeng. 2008, 99, doi: 10.1002/bit.21598, incorporated by reference. Similarly, expression of an antibody in CHO or HEK293 expression platform in the presence of swainsonine will lead to inhibition of α-mannosidase II, thereby generating only the hybrid form of the antibody, as for example reported by Kanda et al., Glycobiology 2006, 17, 104-118, incorporated by reference. A method to generate an antibody with reduced fucosylation is by expression of the antibody in a mammalian expression platform in the presence of a fucosyltransferase inhibitor, for example 6,6,6-trifluorinated derivatives of fucose (fucostatin I and fucostatin II), as reported by Allen et al, ACS Chem. Biol. 2016, 11, 2734-2743, incorporated by reference, or for example 6-modified or 2-modified derivatives of fucose, such as 6-acetylene fucose or 2-fluorofucose, as reported by Rillahan et al., Nat. Chem. Biol. 2012, 8, 661-668 and Okeley et al. Proc. Nat. Acad. Sci. 2013, 110, 5404-5409, both incorporated by reference, or for example by 6-fluoroderivatives of mannose such as Fucotrim I or Fucotrim II, as reported by Pijnenborg et al., ChemRXiv 2020, doi: 10.26434/chemrxiv.13082138.v1, incorporated by reference. In each of the above case, acylated versions of the fucosyltransferase are preferably employed for improved cellular uptake by passive diffusion across the cell membrane.

Abrogation of binding to Fc-gamma receptor can be achieved in various ways, for example by specific mutations in the antibody (specifically the Fc-fragment) or by removal of the N-glycan that is naturally present in the Fc-fragment (CH2 domain, around N297). Glycan removal can be achieved by genetic modification in the Fc-domain, e.g. a N297Q mutation or T299A mutation, or by enzymatic removal of the glycan after recombinant expression of the antibody, using for example PNGase F or an endoglycosidase. For example, endoglycosidase H is known to trim high-mannose and hybrid glycoforms, while endoglycosidase S is able to trim complex type glycans and to some extent hybrid glycan. Endoglycosidase S2 is able to trim both complex, hybrid and high-mannose glycoforms. Endoglycosidase F2 is able to trim complex glycans (but not hybrid), while endoglycosidase F3 can only trim complex glycans that are also 1,6-fucosylated. Another endoglycosidase, endoglycosidase D is able to hydrolyse Man5 (M5) glycan only. An overview of specific activities of different endoglycosidases is disclosed in Freeze et al. in Curr. Prot. Mol. Biol., 2010, 89:17.13A.1-17, incorporated by reference herein. An additional advantage of deglycosylation of proteins for therapeutic use is the facilitated batch-to-batch consistency and significantly improved homogeneity.

Based on the adverse influence of binding to Fc-gamma receptors, multiple ADCs in the clinic are generated Fc-silent: antibodies that are no longer able to bind to Fc-gamma receptors. For example MED14276, a HER²-binding biparatopic ADC with tubulysin payload (AZ13599185), has multiple mutations in the Fc region, L234F, S239C, and S442C. The two engineered cysteine residues per heavy chain (S239C and S442C) enable site-specific conjugation of AZ13599185 to the antibody via a maleimidocaproyl linker, resulting in a biparatopic ADC with a drug-to-antibody ratio of 4. The mutation L234F in combination with the S239C mutation reduced Fc-gamma receptor binding to minimize the FcgR-mediated, HER²-independent uptake of ADC by normal tissues, thereby reducing off-target toxicity such as thrombocytopenia. Similarly, MGTA-117, an ADC based on c-KIT/CD117-targeted Fc-silent antibody for the transplant setting and conjugated to amanitin, is being developed for patients undergoing immune reset through either autologous or allogeneic stem cell transplant. In addition, insertions of cysteine before and after S239 (i.e., C238i and C239i) showed abolition of antibody-dependent cellular cytotoxicity (ADCC) due to non-binding of Fc gamma receptor IIIA (FcγRIIIA), as reported by Travis Gallagher et al., Pharmaceutics 2019, 546, doi: 10.3390/pharmaceutics11100546.

Besides the negative impact associated with the binding of ADCs to Fc-gamma receptors, it also clear that in various cases, retention of Fc-effector functions related to binding to Fc-gamma receptor can contribute positively to the efficacy of the drug, as for example in case of Kadcyla, Trodelvy, Enhertu and mirvetuximab soravtansine. The present invention provides in the need for antibody-conjugates that exhibit binding to Fc-gamma receptors, in particular those conjugates that are conjugated via the glycan of the antibody.

### SUMMARY OF THE INVENTION

The inventors have found that antibody conjugates, which are conjugated via the glycan of the antibody, can have effector function, i.e. by binding to a Fc-gamma receptor, while it was considered in the art that such antibody conjugates lose the effector function of native antibodies. In other words, the antibody conjugates according to the invention are capable of activating immune cells. More specifically, the inventors found that a glycan of structure -GlcNAc(Fuc)_{b}-(G)ₑ-Su-, wherein G and Su are monosaccharides, b=0 or 1 and e is an integer in the range of 4-10, maintain effector function which was considered lost for this class of antibody conjugates. The inventors have for the first time demonstrated binding to Fc-gamma receptor for antibody conjugates conjugated via the glycan of the antibody.

The present invention concerns a method for activation of an immune cell employing these antibody conjugates. The invention further concerns novel antibody conjugates which have effector function. In a further aspect, the invention a process for making these antibody conjugates, a pharmaceutical composition comprising the same, and the medical use thereof.

### DESCRIPTION OF THE FIGURES

FIG. 1 shows a representative (but not comprehensive) set of functional groups (F) that can be introduced into a glycoprotein by engineering, by chemical modification, or by enzymatic means, which upon metal-free click reaction with a complementary reactive group Q lead to connecting group Z. Functional group F may be introduced into a (glyco)protein at any position of choice by engineering, chemical or enzymatic modification. The pyridazine connecting group (bottom line) is the product of the rearrangement of the tetraazabicyclo[2.2.2]octane connecting group, formed upon reaction of tetrazine with alkyne, with loss of N₂. Connecting groups Z of structure (1a)-(1j) are preferred connecting groups to be used in the present invention.

FIG. 2 shows cyclooctynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring.

FIG. 3 shows several structures of derivatives of UDP sugars of galactosamine, which may be modified with e.g. a 3-mercaptopropionyl group (2a), an azidoacetyl group (2b), or an azidodifluoroacetyl group (2c) at the 2-position, or with an azido group at the 6-position of N-acetyl galactosamine (2d) or with a thiol group at the 6-position of N-acetyl galactosamine (2e). The monosaccharide (i.e. with UDP removed) are preferred moieties Su to be used in the present invention.

FIG. 4 shows the general scheme for preparation of antibody-drug conjugates by reaction of a monoclonal antibody (in most cases a symmetrical dimer) containing an x number of functionalities F. By incubation of antibody-(F)ₓ with excess of a linker-drug construct (Q-spacer-linker-payload) a conjugate is obtained by reaction of F with Q, forming connecting group Z.

FIG. 5 shows the general scheme for preparation of antibody-drug conjugates by remodeling/conjugation of the glycan of a monoclonal antibody based on (a) enzymatic trimming to core GlcNAc, (b) enzymatic transfer of an azido-sugar and (c) metal-free click chemistry with a BCN-linker-drug. The azide of the azido-sugar may be on any position in the carbohydrate, preferably the 2-position or the 6-position. Instead of azide, the sugar can also harbour any of the other functional moieties F from FIG. 1.

FIG. 6 depicts the cell expression pattern of Fc gamma receptors (FcγRs) on various immune cells.

FIG. 7 depicts the structures of representative BCN-linker-payloads with MMAE (3a and 3b), PBD (4) or exatecan (5a and 5b), suitable for conjugation to sugar-remodeled antibodies containing azide functionality or others (tetrazine, 1,2-quinone, sydnone, etc).

FIG. 8 shows the N-glycosylation pathway as it takes place in the Golgi, starting from high-mannose N-glycan M7—M9 (A), trimming by mannosidases to M5 (B), attachment of N-acetylglucosamine (GlcNAc) to give hybrid N-glycan M5G0 (C), which may be fucosylated to give M5G0F, further trimming by mannosidases to truncated glycan M3G0 (D), which may be fucosylated to give M3G0F, attachment of GlcNAc to give complex glycan G0 (E), which may be fucosylated to give G0F, which may be chain-extended by attachment of galactose (Gal) to give G1, which may be fucosylated to give G1F, and/or alternatively may be further chain-extended with sialic acid (Sial/Neu5Ac) to give S1G1 or S1G1F (all depicted as F). The additional galactose and optional sialic acid chain-extension may also take place at the other GlcNAc. The G0(F) glycoform may be further modified by attachment of GlcNAc to the core mannose to give bisected glycan G0(F)B (G). The N-glycosylation pathway may be interrupted by the specific mannosidase inhibitors such as kifunensin or swainsonine (open arrows).

FIG. 9 shows the binding of different ADCs to FcγRI (CD64) and FcγRIIIA (CD16A, 176Val and 176Phe mutant). IgG4 is used as negative control and trastuzumab as positive control. Legend: SiteClick™=ADC based on conjugation of 3a (MMAE) to 6-N₃-GalNAc, attached to terminal GlcNAc in G0(F) glycoform; GlycoConnect™=ADC based on conjugation of 3a (MMAE) to 6-N₃-GalNAc, attached to core GlcNAc (after trimming with endoglycosidase); Swainsonine=ADC based on conjugation of 3a (MMAE) to 6-N₃-GalNAc, attached to GlcNAc in M5(F) glycoform of antibody expressed in presence of inhibitor swainsonine (see FIG. 8C); Bisected ADC=ADC based on conjugation of 3a (MMAE) to GlcNAz, attached to mannose M1 in G0(F) glycoform; Kifunensin=ADC based on conjugation of 3a (MMAE) to GlcNAz, attached to mannose on M5 glycoform of antibody expressed in presence of inhibitor kifunensin (see FIG. 8A); * No binding observed.

FIG. 10 shows the binding of MMAE-based ADCs to FcγIIIA (CD16A) relative to trastuzumab (100%). GlycoConnect™ ADC shows no binding to FcγIIIA, whereas SiteClick™ (6-azidoGalNAc) ADC, Bisected ADC and Sialic acid ADC have most of the glycan intact and hence show binding to FcγIIIA.

FIG. 11 shows survival plots of MMAE-based ADCs on BT474 (HER²-positive) and MDA-MB-231 (HER²-negative) cell lines. A clear dose response curve is seen for all ADCs on BT474 cells, whereas no cytotoxicity is observed for the MDA-MB-231 cells.

FIG. 12 shows survival plots of exatecan-based ADCs on BT474, N87 and MDA-MB-231. A clear dose response curve is seen for all ADCs on BT474 and N87 cells, whereas no cytotoxicity is observed for the MDA-MB-231 cells.

FIG. 13 shows the in vitro activation of FcγIIIA. Effector cells are mixed with HER²-positive or negative cells and dilutions of MMAE-based ADCs are added. The plot for HER²-positive cells clearly shows that all ADCs except for the GlycoConnect™ ADC show increasing luminescent signal at increasing concentrations, thereby indicating that the ADCs that bind FcγIIIA (e.g. in ELISA) do also activate the receptor in vitro. The activation was specific to HER²-positive cells only.

### DETAILED DESCRIPTION OF THE INVENTION CL Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as R and S stereoisomers. Unless stated otherwise, the description of any compound in the description and in the claims is meant to include both the individual R and the individual S stereoisomers of a compound, as well as mixtures thereof. When the structure of a compound is depicted as a specific S or R stereoisomer, it is to be understood that the invention of the present application is not limited to that specific S or R stereoisomer.

The compounds disclosed in this description and in the claims may further exist as R and S stereoisomers. Unless stated otherwise, the description of any compound in the description and in the claims is meant to include both the individual R and the individual S stereoisomers of a compound, as well as mixtures thereof. When the structure of a compound is depicted as a specific S or R stereoisomer, it is to be understood that the invention of the present application is not limited to that specific S or R stereoisomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically acceptable” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multi-specific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen or Fc-gamma receptor and not with the multitude of other antigens or Fc-gamma receptors. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen or receptor (e.g., BSA, casein) other than the predetermined antigen or a closely-related antigen.

The term “activation” in the context of an immune cells refers to the enhancement of a signalling pathway of the immune cell. As a result of the activation, the immune cell will be induced to undergo proliferation, to excrete immunoglobulins or cytokines or other immunomodulating molecules.

The term “inhibition” in the context of an immune cells refers to the reduction of a signalling pathway of the immune cell. As a result of the inhibition, the immune cell will be less inclined to undergo proliferation, to excrete immunoglobulins or cytokines or other immunomodulating molecules.

The term “substantial” or “substantially” is herein defined as a majority, i.e. >50% of a population, of a mixture ora sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody conjugate, an antibody and a payload are covalently connected to each other via a linker. A linker may comprise one or more linkers and spacer-moieties that connect various moieties within the linker.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer but branches off from the chemical spacer connecting the antibody and the payload.

A “conjugate” is herein defined as a compound wherein an antibody is covalently connected to a payload via a linker. A conjugate comprises one or more antibodies and/or one or more payloads.

The term “payload” refers to the moiety that is covalently attached to a targeting moiety such as an antibody, but also to the molecule that is released from the conjugate upon uptake of the protein conjugate and/or cleavage of the linker. Payload thus refers to the monovalent moiety having one open end which is covalently attached to the targeting moiety via a linker and also to the molecule that is released therefrom.

The terms “tyrosinase” and “(poly)phenol oxidase” refer to an enzyme that is capable of catalysing the ortho-hydroxylation of a monophenol moiety to an ortho-dihydroxybenzene (catechol) moiety, followed by further oxidation of the ortho-dihydroxybenzene moiety to produce an ortho-quinone (1,2-quinone) moiety.

The term “deglycosylation” refers to the treatment of an N-glycoprotein with an amidase to remove the entire glycan, i.e. by enzymatic hydrolysis of the amide bond between the amino acid, usually asparagine, of the protein and the first monosaccharide, usually GlcNAc, at the reducing end of the glycan.

The term “deglycosylated protein” refers to an N-glycoprotein that has been treated with an amidase to remove the entire glycan, i.e. by enzymatic hydrolysis of the amide bond between the amino acid, usually asparagine, of the protein and the first monosaccharide, usually GlcNAc, at the reducing end of the glycan.

The term “trimming” refers to the treatment of an N-glycoprotein with an endoglycosidase to hydrolyse the glycosidic bond between the first monosaccharide, usually GlcNAc, at the reducing end of the glycan, which is attached to an amino acid, usually asparagine, and the second monosaccharide, usually GlcNAc.

The term “trimmed protein” refers to an N-glycoprotein that has been treated with an endoglycosidase to hydrolyse the glycosidic bond between the first monosaccharide, usually GlcNAc, at the reducing end of the glycan, which is attached to an amino acid, usually asparagine, and the second monosaccharide, usually GlcNAc.

The terms “GlcNAz” and “GalNAz” refer to derivatives of GlcNAc and GalNAc, respectively, wherein the N-acetyl group is replaced by an N-azidoacetyl group.

The term “sialic acid” and “neuraminic acid” and “Neu5Ac” refer to the C-9 sugar N-acetyl-5-amino-3,5-dideoxy-D-glycero-D-galacto-non-2-ulosonic acid and are used interchangeably.

### The Invention

The inventors have found that antibody conjugates, which are conjugated via the glycan of the antibody, can have effector function, i.e. binding to Fc-gamma receptor, while it was considered in the art that such antibody conjugates lost the effector function of unconjugated antibodies. In other words, these antibody conjugates are capable of activating immune cells. More specifically, the inventors found that a glycan of structure -GlcNAc(Fuc)_{b}-(G)ₑ-Su-, wherein G and Su are monosaccharides, b=0 or 1 and e is an integer in the range of 4-10, maintain effector function which was considered lost for this class of antibody conjugates. The inventors have for the first time demonstrated binding to Fc-gamma receptor for antibody conjugates conjugated via the glycan of the antibody.

The present invention concerns a method for activation of an immune cell employing these antibody conjugates. The invention further concerns novel antibody conjugates which have effector function. In a further aspect, the invention concerns a process for making these antibody conjugates, a pharmaceutical composition comprising the same, and the medical use thereof.

Thus, in a first aspect, the invention concerns a method for binding to a cell comprising an Fc-gamma receptor. The process according to the invention comprises contacting the cell with an antibody conjugate, wherein the antibody conjugate has structure (1):

Ab-[(GlcNAc(Fuc)_{b}-(G)ₑ-(Su-(Z-L-(D)ᵣ)ₓ)ₛ]_{y} (1)

wherein:
Ab is an antibody
GlcNAc is an N-acetylglucosamine moiety;
Fuc is a fucose moiety;
b is 0 or 1;
G is a monosaccharide;
e is an integer in the range of 4-10;
Su is a monosaccharide;
Z is a connecting group obtained by a cycloaddition or a nucleophilic reaction;
L is a linker;
D is a payload;
s is 1 or 2;
r is an integer in the range of 1-4;
x is 1 or 2;
y is 2 or 4.

The invention further concerns an antibody conjugate, wherein the antibody conjugate has structure (1):

Ab-[(GlcNAc(Fuc)_{b}-(G)ₑ-(Su-(Z-L-(D)ᵣ)ₓ)ₛ]_{y} (1)

wherein:
Ab is an antibody
GlcNAc is an N-acetylglucosamine moiety;
Fuc is a fucose moiety;
b is 0 or 1;
(G)ₑ is an oligosaccharide of structure (G1):

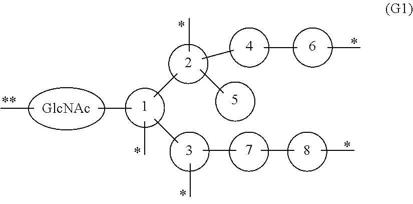

wherein (G)ₑ is connected to GlcNAc(Fuc)b via the bond labelled with ** and to Su via one of the bonds labelled *; and
   (i) (1)=(2)=(3)=(4)=(5)=Man; (6)=(7)=(8)=absent; and (G)ₑ is connected to Su via (3);
   (ii) (1)=(2)=(3)=(4)=(5)=(6)=Man; (7)=(8)=absent; and (G)ₑ is connected to Su via (3);
   (iii) (1)=(2)=(3)=(4)=Man; (5)=(6)=(7)=(8)=absent; and (G)ₑ is connected to Su via (3);
   (vii) (1)=(2)=(3)=Man; (4)=(7)=GlcNAc; (5)=(8)=absent; (6)=Gal; and (G)ₑ is connected to Su via (1);
   (viii) (1)=(2)=(3)=Man; (4)=(7)=GlcNAc; (5)=(6)=(8)=absent; and (G)ₑ is connected to Su via (1);
   (ix) (1)=(2)=(3)=Man; (4)=(7)=GlcNAc; (5)=(8)=absent; (6)=Gal; and (G)ₑ is connected to Su via (3);
   (x) (1)=(2)=(3)=Man; (4)=(7)=GlcNAc; (5)=(6)=(8)=absent; and (G)ₑ is connected to Su via (3);
e is an integer in the range of 4-10;
Su is a monosaccharide;
Z is a connecting group obtained by a cycloaddition or a nucleophilic reaction;
L is a linker;
D is a payload;
s is 1 or 2;
r is an integer in the range of 1-4;
x is 1 or 2;
y is 2 or 4.

The invention concerns in a first aspect a method wherein an antibody conjugate is contacted with a cell comprising an Fc-gamma receptor, and in a second aspect the antibody conjugate itself. The antibody conjugate according to both aspects is the same, and also referred to as the antibody conjugate according to the invention, except for the definition of (G)ₑ, which is different for the first and the second aspects. Thus, except where clearly indicated, the definition of the antibody conjugate according to the invention applies to all aspects of the invention.

### The Method for Binding to a Cell Comprising an Fc-Gamma Receptor

The first aspect of the invention concerns a method for binding to a cell comprising an Fc-gamma receptor. The binding is preferably to Fc-gamma receptor IA, IIA or IIIA. The method involves contacting an antibody conjugate according to the invention with the cell. The method may occur in vitro or in vivo. In one aspect, the method according to the invention is a therapeutic method, wherein cells, typically immune cells, are bound to in vivo. Also ex vivo methods are covered by the present invention. Upon binding of the antibody conjugate to the Fc-gamma receptor of a cell, typically an immune cell, that cell may become activated, activating the immune system of the subject. The method can thus also be worded as for activating the immune system.

The method according to the present aspect can also be worded as a method for activation of a cell comprising an Fc-gamma receptor or as a method for targeting cells comprising an Fc-gamma receptor, preferably an Fc-gamma receptor IA, IIA or IIIA. The method according to this aspect can also be worded as the use of the antibody conjugate according to the invention for binding to a cell comprising an Fc-gamma receptor, the use of the antibody conjugate according to the invention for activation of a cell comprising an Fc-gamma receptor or as the use of the antibody conjugate according to the invention for targeting cells comprising an Fc-gamma receptor.

Herein, the cell comprising an Fc-gamma receptor is typically a cell expressing an Fc-gamma receptor. Such a cell is typically an immune cell, preferably a human immune cell. Suitable cells include lymphocytes, follicular cells, dendritic cells, natural killer cells, B cells, T cells, macrophages, neutrophils, eosinophils, basophils, platelets and mast cells. The cell is typically comprised in a sample comprising a plurality of cells. The sample may be taken from a present in a subject, typically a human subject. In a particular embodiment, the subject is a cancer patient.

In a preferred embodiment, the binding of the antibody conjugate according to the invention is improved over the binding of the same antibody conjugate but wherein e is 0, or even wherein e is below 4. Similarly, the activation of the immune system is preferably improved over the activation by the same antibody conjugate but wherein e is 0, or even wherein e is below 4. Similarly, the targeting of cells is preferably improved over the targeting of cells by the same antibody conjugate but wherein e is 0, or even wherein e is below 4.

### The Antibody Conjugate

The antibody conjugate according to the invention has structure (1):

Ab-[(GlcNAc(Fuc)_{b}-(G)ₑ-(Su-(Z-L-(D)ᵣ)ₓ)ₛ]_{y} (1)

wherein:
Ab is an antibody
GlcNAc is an N-acetylglucosamine moiety;
Fuc is a fucose moiety;
b is 0 or 1;
G is a monosaccharide;
e is an integer in the range of 4-10;
Su is a monosaccharide;
Z is a connecting group obtained by a cycloaddition or a nucleophilic reaction;
L is a linker;
D is a payload;
s is 1 or 2;
r is an integer in the range of 1-4;
x is 1 or 2;
y is 2 or 4.

The integer y denotes the number of glycans, or more specifically the number of GlcNAc(Fuc)_{b} residues, that are conjugated to one or more payloads D. y=2 or 4, preferably y=2. The integer x denotes the number of connecting groups Z that are connected to monosaccharide Su, which is determined by the number of reactive groups F present on Su(F)ₓ used in the preparation of the antibody conjugate according to the invention. x=1 or 2, preferably x=1. The integer r denotes the number of payloads D that are connected to a single linker L. The linker may be linear, having only one occurrence of D connected to it, or may contain one or more branching points to connect up to 4 occurrences of D to the same connecting group Z. Preferably, r is 1 or 2. Integer s denotes the number of monosaccharides Su connected to glycan (G)ₑ. s=1 or 2, preferably s=1.

### The Antibody Ab

The antibody is preferably a monoclonal antibody, more preferably selected from the group consisting of IgA, IgD, IgE, IgG and IgM antibodies. Even more preferably Ab is an IgG antibody.

The IgG antibody may be of any IgG isotype, such as IgG1, IgG2, Igl3 or IgG4. Preferably, the antibody is a full-length antibody, but Ab may also be a Fc fragment. The antibody typically has an N-glycosylation site at asparagin at (or around) position 297 (Kabat numbering).

### The Glycan GlcNAc(Fuc)_{b}-(G)ₑ

The antibody conjugate according to the invention has a glycan of structure -GlcNAc(Fuc)_{b}-(G)ₑ, to which monosaccharide Su is added. Su is a functionalized monosaccharide, comprising x reactive groups F (prior to conjugation) or x connecting groups Z (after conjugation). Hence, Su can be viewed as a monosaccharide derivative, and is further defined below. In view of the monosaccharide core structure of Su, it could be seen as part of the glycan. However, the glycan of structure -GlcNAc(Fuc)_{b}-(G)ₑ originates from the original glycan of the antibody, to which Su is attached (see also step (c) of the process of the third aspect of the invention).

The -GlcNAc(Fuc)_{b}-(G)ₑ of the glycan thus typically originates from the original antibody, wherein GlcNAc is an N-acetylglucosamine moiety and Fuc is a fucose moiety. Fuc is typically bound to GlcNAc via an α-1,6-glycosidic bond. Normally, antibodies may (b=1) or may not be fucosylated (b=0). Although the inventors found that both fucosylated, with b=1, and non-fucosylated, with b=0, may exhibit effector function, the greatest effects were observed for non-fucosylated antibody conjugates. It is thus preferred that b=0. The GlcNAc residue may also be referred to as the core-GlcNAc residue and is the monosaccharide that is directly attached to the peptide part of the antibody.

(G)ₑ is an oligosaccharide fraction comprising e monosaccharide residues G, wherein e is an integer in the range of 4—10. (G)ₑ is connected to the GlcNAc moiety of GlcNAc(Fuc)_{b}, typically via a β-1,4 bond. In a preferred embodiment, e is 5, 6 or 7. Although any monosaccharide that may be present in a glycan may be employed as G, each G is preferably individually selected from the group consisting of galactose, glucose, N-acetylgalactosamine, N-acetylglucosamine, mannose and N-acetylneuraminic acid. More preferred options for G are galactose, N-acetylglucosamine and mannose.

The (G)ₑ fragment is key in the present invention and determines whether the antibody conjugate binds to the Fc-gamma receptor or not. Antibody conjugates having e below 4 show no or hardly any binding to the Fc-gamma receptor, while antibody conjugates having e in the range of 4-10 do bind to the Fc-gamma receptor.

In a preferred embodiment, (G)ₑ is connected to GlcNAc(Fuc)_{b} via a GlcNAc monosaccharide residue. Preferably, (G)ₑ is according to structure (G1):

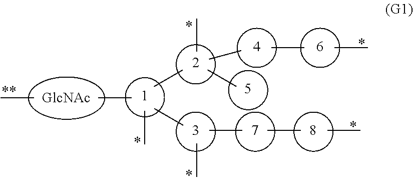

wherein:
(G)ₑ is connected to GlcNAc(Fuc)_{b} via the bond labelled with ** and to Su via one of the bonds labelled *;
monosaccharide (1) is Man;
monosaccharide (2) is Man or absent;
monosaccharide (3) is Man;
monosaccharide (4) is Man, GlcNAc or absent;
monosaccharide (5) is Man or absent;
monosaccharide (6) is Man, Gal or absent;
monosaccharide (7) is GlcNAc or absent;
monosaccharide (8) is Gal or absent.

The GlcNAc residue and the three Man residues (1), (2) and (3) form the core of the glycan. All other monosaccharide residues, as well as mannose residue (2), may be absent. Notably, when monosaccharide (6) is absent, monosaccharide (4) may bound directly to Su via the bond labelled with *. When monosaccharide (4) is absent, monosaccharide (6) is bound directly to (6), unless monosaccharide (6) is also absent, in which case monosaccharide (2) may be bound directly to Su via the bond labelled with *. Monosaccharide (2) may also be bound directly to Su via the bond labelled with * in case monosaccharides (4) and/or (6) are present. On the other hand, monosaccharide (7) may be bound directly to Su via the bond labelled with * only in case monosaccharide (8) is absent. Monosaccharide (2) is Man or absent, preferably (2)=Man. Monosaccharide (4) is Man, GlcNAc or absent, preferably (4)=Man. Monosaccharide (6) is Man, Gal or absent, preferably (6)=absent. Monosaccharide (7) is GlcNAc or absent, preferably (7)=GlcNAc. Monosaccharide (8) is Gal or absent, preferably (8)=absent.

In the structure (G1), one or two bonds labelled with * may be connected to Su. Thus, the glycan (G)ₑ may bear two occurrences of Su (s=2). This may for example occur when Su is connected to monosaccharide (6) and monosaccharide (8). It is however preferred that the glycan (G)ₑ bears one occurrence of Su, i.e. s=1. Preferred points of attachment to Su when s=1 are monosaccharides (1) and (3).

Especially preferred embodiments of (G)ₑ are according to structure (G1) wherein:
(i) (1)=(2)=(3)=(4)=(5)=Man; (6)=(7)=(8)=absent; and (G)ₑ is connected to Su via (3);
(ii) (1)=(2)=(3)=(4)=(5)=(6)=Man; (7)=(8)=absent; and (G)ₑ is connected to Su via (3);
(iii) (1)=(2)=(3)=(4)=Man; (5)=(6)=(7)=(8)=absent; and (G)ₑ is connected to Su via (3);
(iv) (1)=(2)=(3)=Man; (4)=(5)=(6)=(8)=absent; (7)=GlcNAc; and (G)ₑ is connected to Su via (7);
(v) (1)=(2)=(3)=(4)=Man; (5)=(6)=(8)=absent; (7)=GlcNAc; and (G)ₑ is connected to Su via (7);
(vi) (1)=(2)=(3)=(4)=(5)=Man; (6)=(8)=absent; (7)=GlcNAc; and (G)ₑ is connected to Su via (7);
(vii) (1)=(2)=(3)=Man; (4)=(7)=GlcNAc; (5)=(8)=absent; (6)=Gal; and (G)ₑ is connected to Su via (1);
(viii) (1)=(2)=(3)=Man; (4)=(7)=GlcNAc; (5)=(6)=(8)=absent; and (G)ₑ is connected to Su via (1);
(ix) (1)=(2)=(3)=Man; (4)=(7)=GlcNAc; (5)=(8)=absent; (6)=Gal; and (G)ₑ is connected to Su via (3);
(x) (1)=(2)=(3)=Man; (4)=(7)=GlcNAc; (5)=(6)=(8)=absent; and (G)ₑ is connected to Su via (3);
(xi) (1)=(2)=(3)=Man; (4)=(7)=GlcNAc; (5)=(8)=absent; (6)=Gal; and (G)ₑ is connected to Su via (7);
(xii) (1)=(2)=(3)=Man; (4)=(7)=GlcNAc; (5)=(6)=(8)=absent; and (G)ₑ is connected to Su via (7);
(xiii) (1)=(2)=(3)=Man; (4)=(7)=GlcNAc; (5)=(6)=absent; (8)=Gal; and (G)ₑ is connected to Su via (6) and (8);
(xiv) (1)=(2)=(3)=Man; (4)=(7)=GlcNAc; (5)=(8)=absent; (6)=Gal; and (G)ₑ is connected to Su via (6) and (6);
(xv) (1)=(2)=(3)=Man; (4)=(7)=GlcNAc; (5)=absent; (6)=(8)=Gal; and (G)ₑ is connected to Su via (6) and/or (8), preferably via (6) and (8).
(xvi) (1)=(2)=(3)=Man; (4)=(5)=(6)=(7)=(8)=absent; and (G)ₑ is connected to Su via (3);
(xvii) (1)=(3)=Man; (2)=(4)=(5)=(6)=(7)=(8)=absent; and (G)ₑ is connected to Su via (3);
(xviii) (1)=(3)=Man; (2)=(4)=(5)=(6)=(8)=absent; (7)=GlcNAc; and (G)ₑ is connected to Su via (7).

In case (G)ₑ is according to (i), it is preferred to Su is GlcNAc. In case (G)ₑ is according to (ii), it is preferred to Su is GlcNAc. In case (G)ₑ is according to (iii), it is preferred to Su is GlcNAc. In case (G)ₑ is according to (iv), it is preferred to Su is GalNAc. In case (G)ₑ is according to (v), it is preferred to Su is GalNAc. In case (G)ₑ is according to (vi), it is preferred to Su is GalNAc. In case (G)ₑ is according to (vii), it is preferred to Su is GlcNAc. In case (G)ₑ is according to (viii), it is preferred to Su is GlcNAc. In case (G)ₑ is according to (ix), it is preferred to Su is GlcNAc. In case (G)ₑ is according to (x), it is preferred to Su is GlcNAc. In case (G)ₑ is according to (xi), it is preferred to Su is GalNAc. In case (G)ₑ is according to (xii), it is preferred to Su is GalNAc. In case (G)ₑ is according to (xiii), it is preferred to Su is Neu5Ac. In case (G)ₑ is according to (xiv), it is preferred to Su is Neu5Ac. In case (G)ₑ is according to (xv), it is preferred to Su is Neu5Ac. In case (G)ₑ is according to (xvi), it is preferred to Su is GalNAc. In case (G)ₑ is according to (xvii), it is preferred to Su is GalNAc. In case (G)ₑ is according to (xviii), it is preferred to Su is GalNAc.

In an especially preferred embodiment, (G)ₑ is according to option (i), (ii), (iii), (vii), (viii), (ix) or (x). In an alternative preferred embodiment, (G)ₑ is according to (iii) or (iv). Most preferably, (G)ₑ is according to (iii).

A preferred embodiment of structure (G1) is (G)ₑ according to structure (G2):

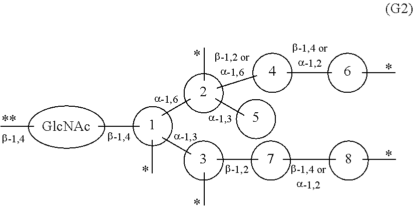

wherein:
(G)ₑ is connected to GlcNAc(Fuc)_{b} via the bond labelled with ** and to Su via one of the bonds labelled *;
monosaccharide (1) is Man;
monosaccharide (2) is Man or absent;
monosaccharide (3) is Man;
monosaccharide (4) is Man, GlcNAc or absent;
monosaccharide (5) is Man or absent;
monosaccharide (6) is Man, Gal or absent;
monosaccharide (7) is GlcNAc or absent;
monosaccharide (8) is Gal or absent.

In structure (G2), the bonding between the individual monosaccharides is specified in case both monosaccharides are present. In case one or both of the monosaccharides is absent, the bond logically is also absent. All further preferred embodiments specified for structure (G1) equally apply to structure (G2).

For the antibody conjugate according to the second aspect of the invention, (G)ₑ is according to structure (G1), preferably according to structure (G2), as defined above, wherein
(i) (1)=(2)=(3)=(4)=(5)=Man; (6)=(7)=(8)=absent; and (G)ₑ is connected to Su via (3);
(ii) (1)=(2)=(3)=(4)=(5)=(6)=Man; (7)=(8)=absent; and (G)ₑ is connected to Su via (3);
(iii) (1)=(2)=(3)=(4)=Man; (5)=(6)=(7)=(8)=absent; and (G)ₑ is connected to Su via (3);
(vii) (1)=(2)=(3)=Man; (4)=(7)=GlcNAc; (5)=(8)=absent; (6)=Gal; and (G)ₑ is connected to Su via (1);
(viii) (1)=(2)=(3)=Man; (4)=(7)=GlcNAc; (5)=(6)=(8)=absent; and (G)ₑ is connected to Su via (1);
(ix) (1)=(2)=(3)=Man; (4)=(7)=GlcNAc; (5)=(8)=absent; (6)=Gal; and (G)ₑ is connected to Su via (3);
(x) (1)=(2)=(3)=Man; (4)=(7)=GlcNAc; (5)=(6)=(8)=absent; and (G)ₑ is connected to Su via (3).

All further preferred embodiments specified for structures (G1) and (G2) equally apply to the antibody conjugate according to the second aspect of the invention.

### Monosaccharide Su

Su is a monosaccharide residue that is attached to (G)ₑ. Su is further connected to one or two instances of Z (x=1 or 2), preferably to one instance of Z. In other words, x is preferably 1. Z is formed by the conjugation reaction between F (located on Su) and Q (connected to D via L). Monosaccharide Su is thus functionalized with one or two occurrences of F (or Z after conjugation). In that respect, one or two hydrogen atoms or hydroxyl moieties of the monosaccharide residue Su may be replaced by F (or Z), in which case Su is still referred to as a monosaccharide.

Su may be any monosaccharide that can normally be attached to a glycan, and is preferably selected from galactose, glucose, N-acetylgalactosamine, N-acetylglucosamine and N-acetylneuraminic acid, preferably wherein Su is N-acetylgalactosamine, N-acetylglucosamine or N-acetylneuraminic acid. In an alternative embodiment, Su is selected from galactose, glucose, N-acetylgalactosamine and N-acetylglucosamine. Most preferably, Su is N-acetylgalactosamine or N-acetylglucosamine.

These monosaccharides are functionalized with one or two occurrences of F or Z. Preferably, these functionalizations occur at the 2 and/or 6 position of the monosaccharide, more preferably at the 2 or the 6 position. In case Su=N-acetylneuraminic acid, the preferred functionalized positions are positions 5 and/or 9, more preferably at the 5 or the 9 position.

Su(F)ₓ may for example be selected from 2-(C(O)(CH₂)ₚF*)-2-deoxy-galactose, 2-(C(O)(CH₂)ₚF*)-2-deoxy-glucose, 2-F*-difluoroacetamido-2-deoxy-galactose, 6-F*-6-deoxy-galactose, 6-F*-6-deoxy-2-acetamidogalactose, 4-F*-4-deoxy-2-acetamidogalactose, 6-F*-6-deoxy-2-F*-acetamido-2-deoxygalactose, 6-F*-6-deoxy-glucose, 6-F*-6-deoxy-2-acetamido-glucose, 4-F*-4-deoxy-2-acetamidoglucose and 6-F*-6-deoxy-2-(F*-acetamido)-2-deoxyglucose. Herein, p is an integer in the range of 0-5. In one embodiment, p=0-5for F*=thiol and p=1 for F*=azide. The 2-(C(O)(CH₂)ₚF*)-2-deoxy-galactose is preferably 2-(F*-acetamido)-2-deoxy-galactose. The 2-(C(O)(CH₂)ₚF*)-2-deoxy-glucose is preferably 2-(F*-acetamido)-2-deoxyglucose. Preferably, Su(F)ₓ is selected from from the group consisting of 2-F*-acetamido-2-deoxy-galactose, 6-F*-6-deoxygalactose and 6-F*-6-deoxy-2-acetamidogalactose, most preferably Su(F)ₓ is 6-F*-6-deoxy-2-acetamidogalactose. In order to avoid confusion with the possible presence of fluorine substituents, reactive group F is here denoted with F*. Reactive group F is further defined below.

In a preferred embodiment, wherein F is an azide, the Su(F)ₓ may for example be selected from 2-azidoacetamido-2-deoxy-galactose (GalNAz), 2-azidodifluoroacetamido-2-deoxy-galactose (F₂-GalNAz), 6-azido-6-deoxygalactose (6-AzGal), 6-azido-6-deoxy-2-acetamidogalactose (6-AzGalNAc or 6-N₃-GalNAc), 4-azido-4-deoxy-2-acetamidogalactose (4-AzGalNAc), 6-azido-6-deoxy-2-azidoacetamido-2-deoxygalactose (6-AzGalNAz), 2-azidoacetamido-2-deoxyglucose (GlcNAz), 6-azido-6-deoxyglucose (6-AzGlc), 6-azido-6-deoxy-2-acetamidoglucose (6-AzGlcNAc), 4-azido-4-deoxy-2-acetamidoglucose (4-AzGlcNAc) and 6-azido-6-deoxy-2-azidoacetamido-2-deoxyglucose (6-AzGlcNAz). Preferably, Su(F)ₓ is selected from from the group consisting of GalNAz, 6-AzGal and 6-AzGalNAc, most preferably Su(F)ₓ is 6-AzGalNAc.

In an alternative preferred embodiment, wherein F is a thiol, the nucleotide sugar is preferably selected from 2-(C(O)(CH₂)ₚSH)-2-deoxy-galactose, 2-(C(O)(CH₂)ₚSH)-2-deoxy-glucose, 6-thio-6-deoxygalactose (6-thio-Gal), 6-thio-6-deoxy-2-acetamidogalactose (6-thio-GalNAc), 6-thio-6-deoxyglucose (6-thio-Glc) and 6-thio-6-deoxy-2-acetamido-glucose (6-thioGlcNAc). Herein, p is an integer in the range of 0—5.

### Connecting Group Z

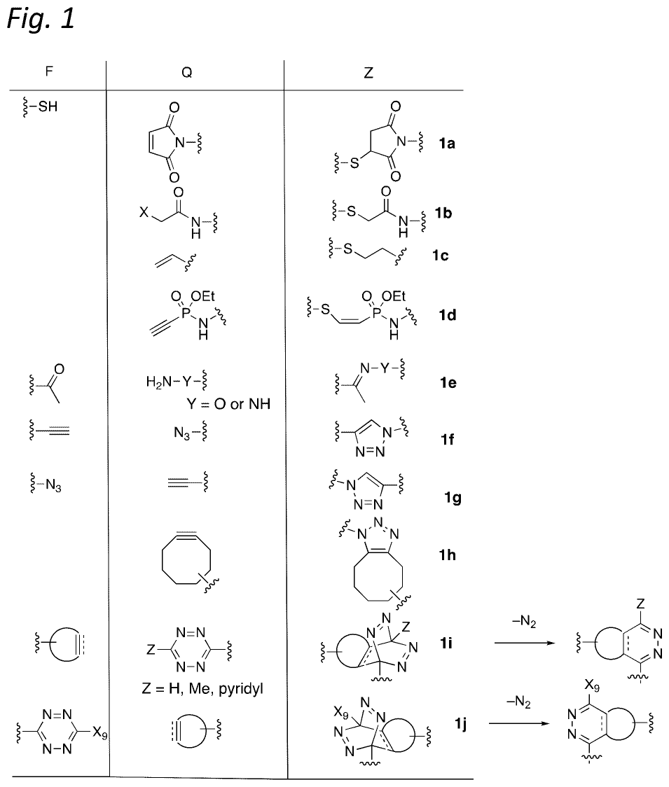

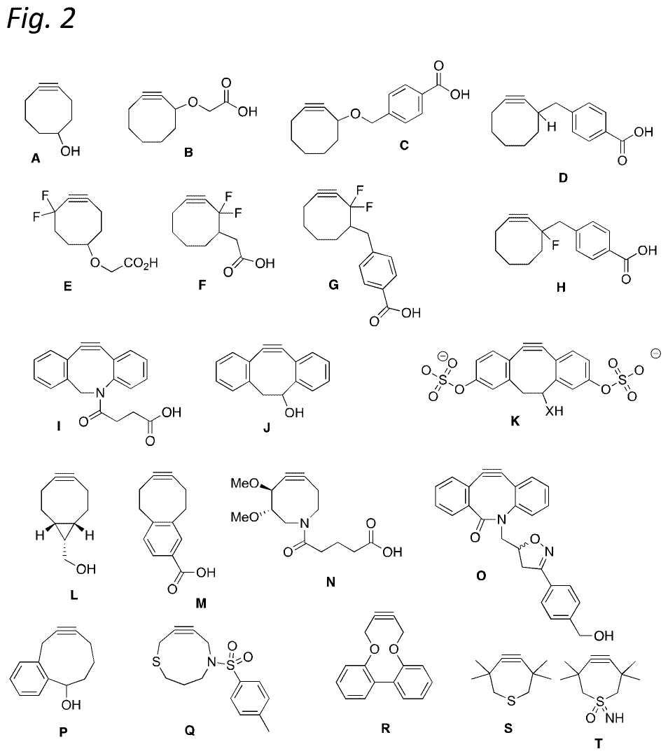

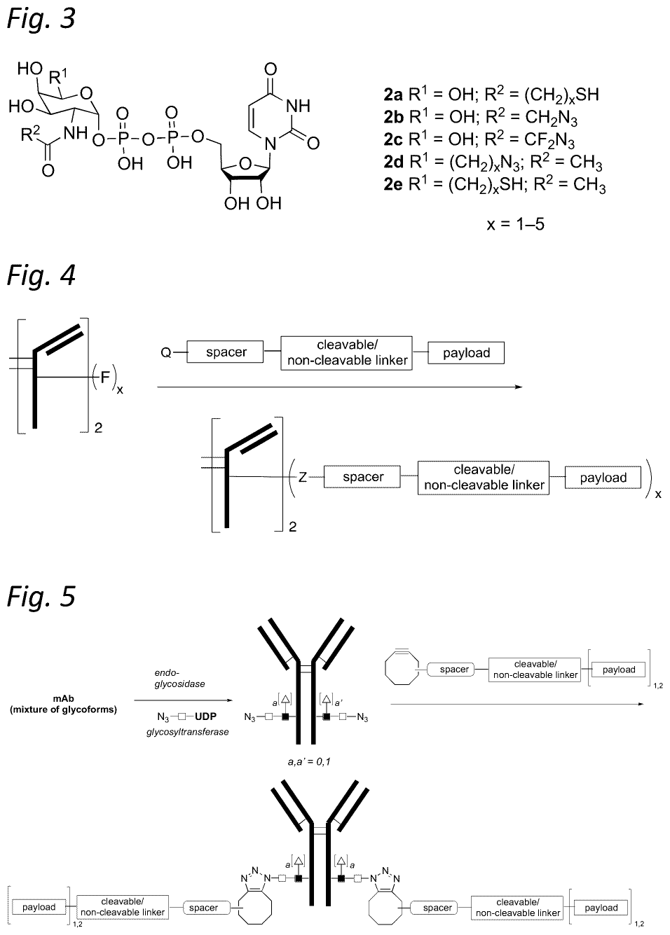

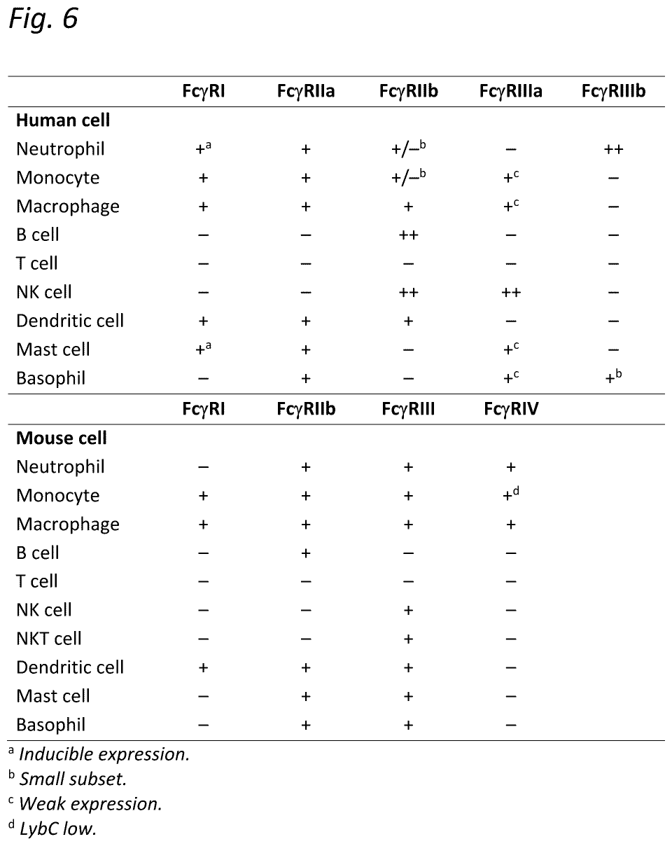

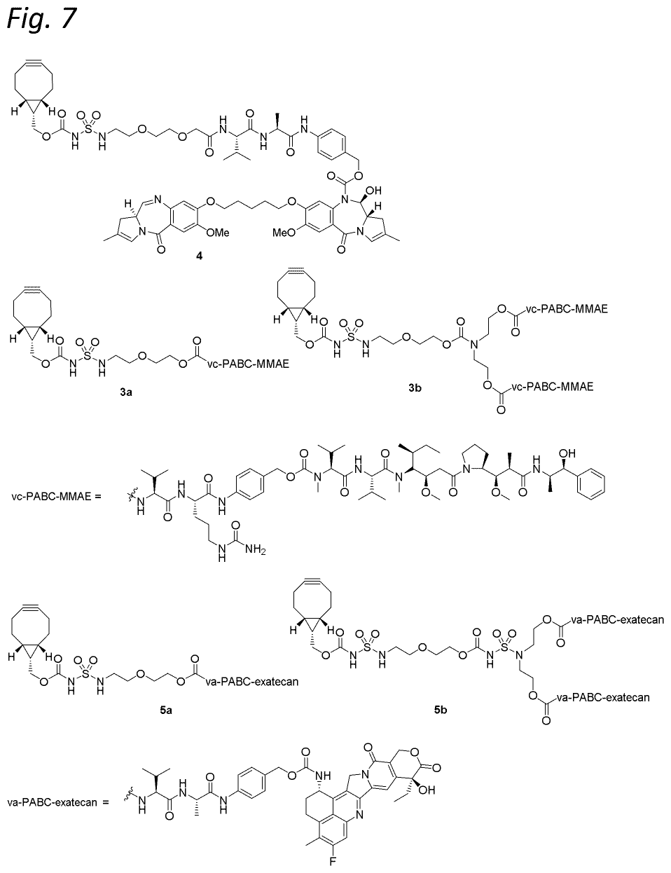

Z is a connecting group. The term “connecting group” refers to a structural element connecting one part of the conjugate and another part of the same bioconjugate. In (1), Z connects antibody Ab (via Su) with the payload D (via L). Connecting group Z is obtained by a cycloaddition or a nucleophilic reaction, preferably wherein the cycloaddition is a [4+2] cycloaddition or a 1,3-dipolar cycloaddition or the nucleophilic reaction is a Michael addition or a nucleophilic substitution. Such a cycloaddition or nucleophilic reaction occurs via a reactive group F, connected to Su, and reactive group Q, connected to D via L. Conjugation reactions via cycloadditions or nucleophilic reactions are known to the skilled person, and the skilled person is capable of selecting appropriate reaction partners F and Q, and will understand the nature of the resulting connecting group Z. Some exemplary options for reactive group Q are provided in FIG. 2, and some exemplary combinations of Q and F, and the corresponding connecting group Z, are provided in FIG. 1. Further guidance is provided in G. T. Hermanson, “Bioconjugate Techniques”, Elsevier, 3rd Ed. 2013 (ISBN:978-0-12-382239-0), in particular in Chapter 3, pages 229-258, incorporated by reference.

In a first preferred embodiment, Z is formed by a cycloaddition. Preferred cycloadditions are a (4+2)-cycloaddition (e.g. a Diels-Alder reaction) or a (3+2)-cycloaddition (e.g. a 1,3-dipolar cycloaddition). Preferably, the conjugation is the Diels-Alder reaction or the 1,3-dipolar cycloaddition. The preferred Diels-Alder reaction is the inverse-electron demand Diels-Alder cycloaddition. In another preferred embodiment, the 1,3-dipolar cycloaddition is used, more preferably the alkyne-azide cycloaddition, and most preferably wherein Q is or comprises an alkyne group and F is an azido group. Cycloadditions, such as Diels-Alder reactions and 1,3-dipolar cycloadditions are known in the art, and the skilled person knowns how to perform them.

Preferably, Z contains a moiety selected from the group consisting of a triazole, a cyclohexene, a cyclohexadiene, a [2.2.2]-bicyclooctadiene, a [2.2.2]-bicyclooctene, an isoxazoline, an isoxazolidine, a pyrazoline, a piperazine, a thioether, an amide or an imide group. Triazole moieties are especially preferred to be present in Z. In one embodiment, Z comprises a (hetero)cycloalkene moiety, i.e. formed from Q comprising a (hetero)cycloalkyne moiety. In an alternative embodiment, Z comprises a (hetero)cycloalkane moiety, i.e. formed from Q comprising a (hetero)cycloalkene moiety. In a preferred embodiment, Z has the structure (Z1):

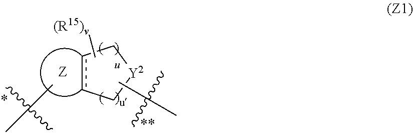

Herein, the bond depicted as [symbol] is a single bond or a double bond. Furthermore:
ring Z is obtained by a cycloaddition, preferably ring Z is selected from (Za)-(Zj) defined below, wherein the carbon atoms labelled with ** correspond to the two carbon atoms of the bond depicted as [symbol] of (Z1) to which ring Z is fused;
R¹⁵ is independently selected from the group consisting of hydrogen, halogen, —OR^{16,} —NO², —CN, —S(O)₂R¹⁶, −S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₇-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S⁽⁺⁾ is a cationic sulphur atom counterbalanced by B⁽⁻⁾, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 8-16;
Ring A is formed by the cycloaddition, and is preferably selected from (Za)-(Zj).

In case the bond depicted as [symbol] is a double bond, it is preferred that u+u′=4, 5, 6, 7 or 8. Preferably, the wavy bond labelled with * is connected to Su and the wavy bond labelled with ** is connected to L.

It is especially preferred that Z comprises a (hetero)cycloalkene moiety, i.e. the bond depicted as [symbol] is a double bond. In a preferred embodiment, Z is selected from the structures (Z2)-(Z20), depicted here below:

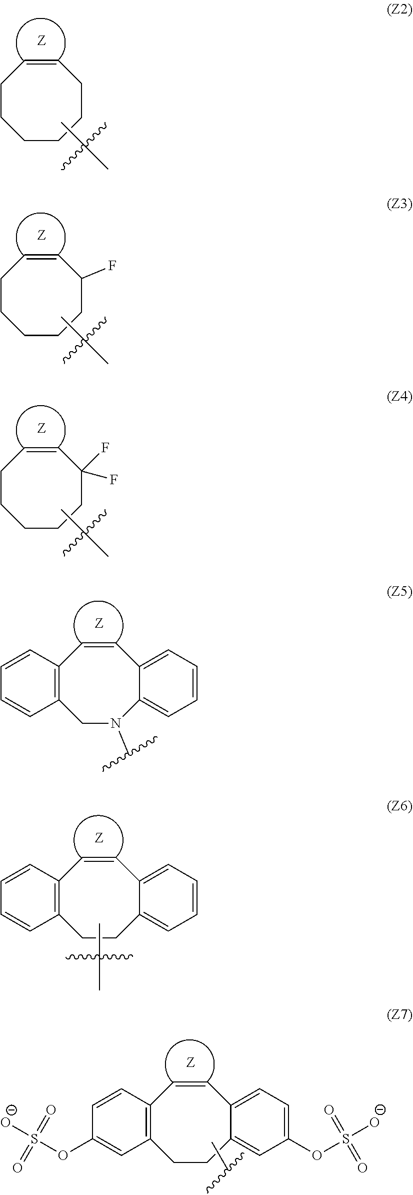

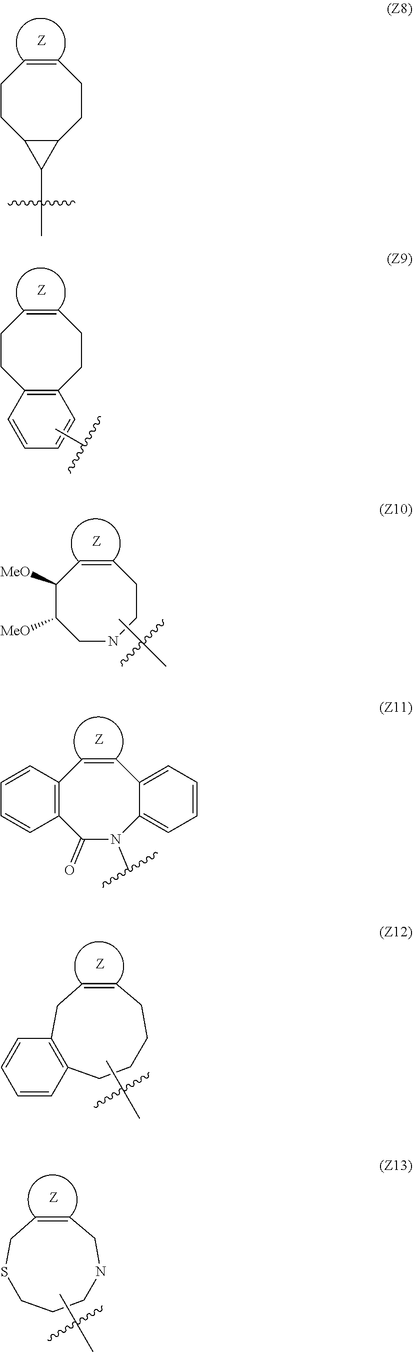

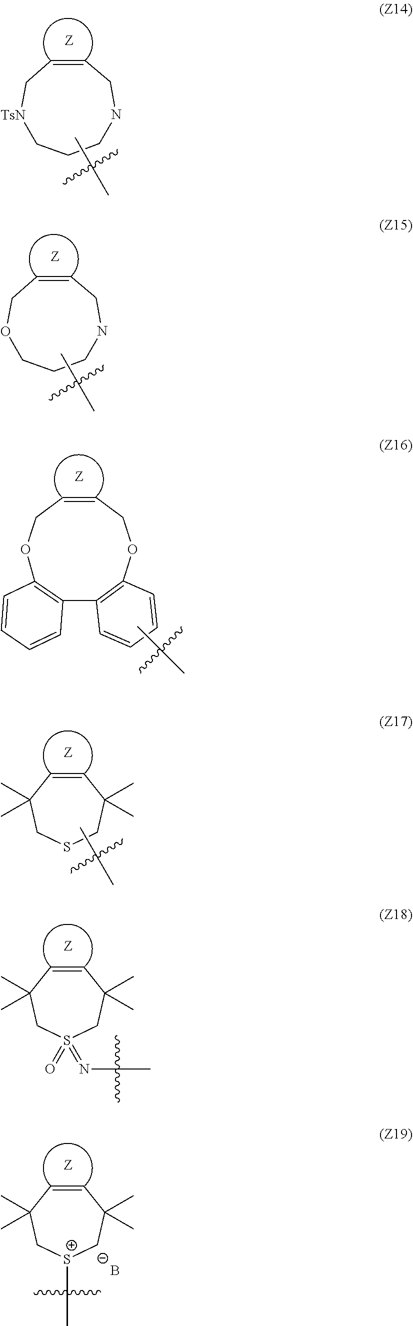

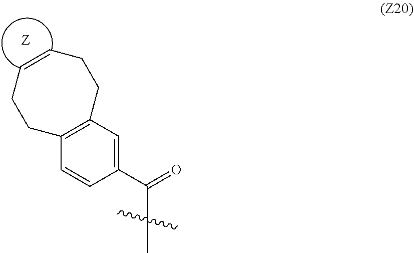 [SMILES: *C(=O)c1ccc2c(c1)CC/C=C\CC2]

Herein, the connection to L is depicted with the wavy bond. B⁽⁻⁾ is an anion, preferably a pharmaceutically acceptable anion. Ring Z is formed by the cycloaddition reaction, and preferably is a triazole, a cyclohexene, a cyclohexadiene, a [2.2.2]-bicyclooctadiene, a [2.2.2]-bicyclooctene, an isoxazoline, an isoxazolidine, a pyrazoline or a piperazine. Most preferably, ring Z is a triazole ring. Ring Z may have the structure selected from (Za)-(Zj) depicted below, wherein the carbon atoms labelled with ** correspond to the two carbon atoms of the (hetero)cycloalkane ring of (Z2)-(Z20) to which ring Z is fused. Since the connecting group Z is formed by reaction with a (hetero)cycloalkyne in the context of the present embodiment, the bound depicted above as [symbol] is a double bond.

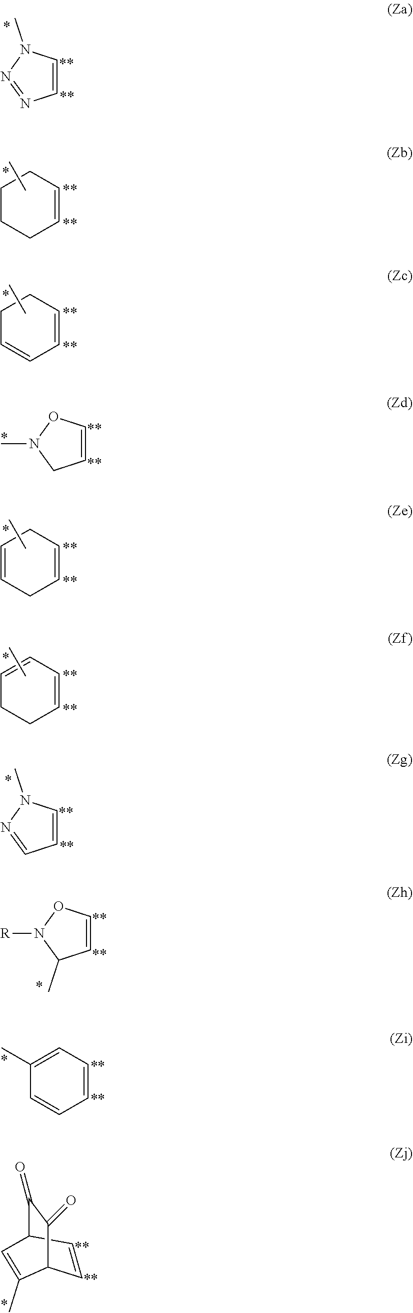

In a further preferred embodiment, Z is selected from the structures (Z21)—(Z38), depicted here below:

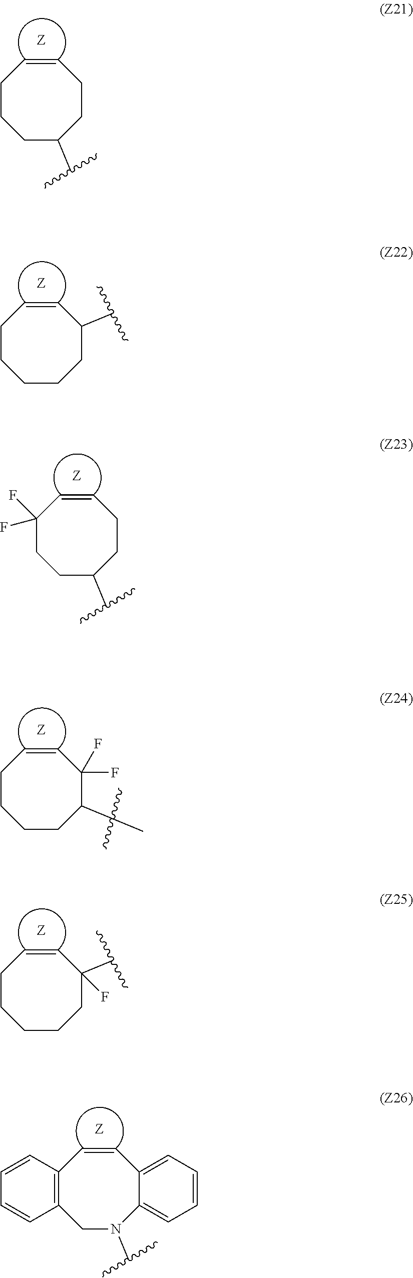 [SMILES: *C1CC/C=C\CCC1 ; *C1/C=C\CCCCC1 ; *C1CC/C=C\C(F)(F)CC1 ; *C1CCCC/C=C\C1(F)F ; *C1(F)/C=C\CCCCC1 ; *N1Cc2ccccc2/C=C\c2ccccc21]

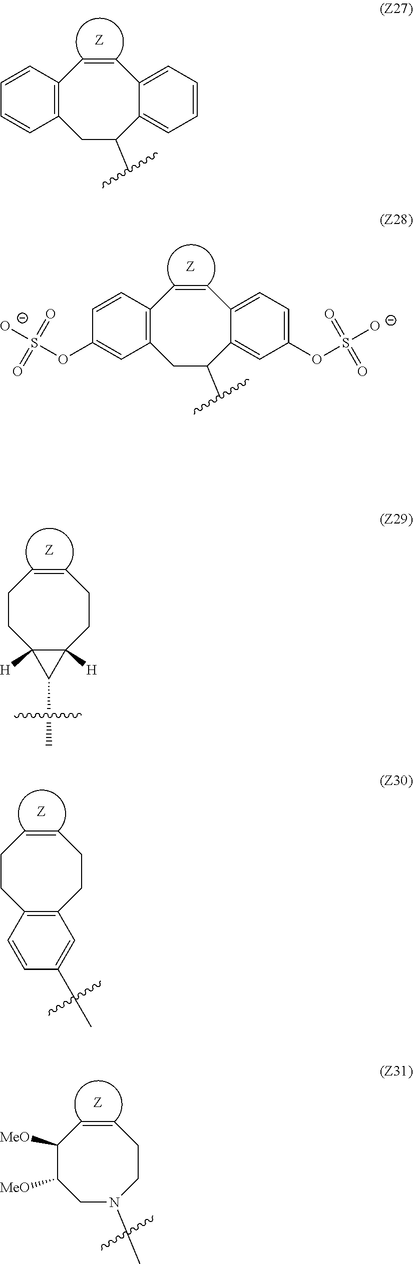

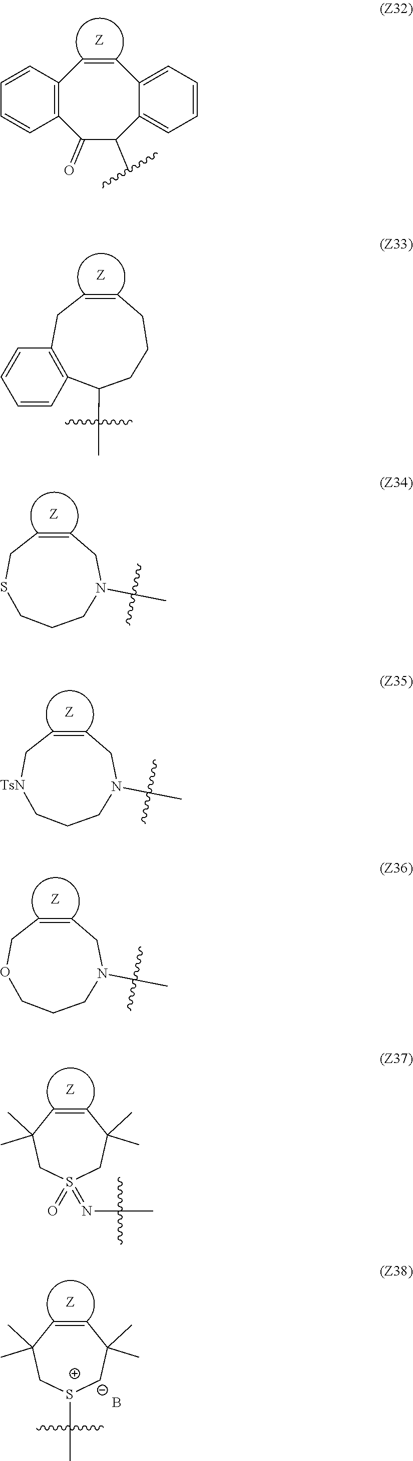

Herein, the connection to L is depicted with the wavy bond. In structure (Z38), B⁽⁻⁾ is an anion, preferably a pharmaceutically acceptable anion. Ring Z is selected from structures (Za)-(Zj), as defined above.

In a preferred embodiment, Z comprises a (hetero)cyclooctene moiety according to structure (Z8), more preferably according to (Z29), which is optionally substituted. In the context of the present embodiment, Z preferably comprises a (hetero)cyclooctene moiety according to structure (Z39) as shown below, wherein V is (CH₂)_{I} and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Z39), I is most preferably 1. Most preferably, Z is according to structure (Z42), defined further below.

In an alternative preferred embodiment, Z comprises a (hetero)cyclooctene moiety according to structure (Z26), (Z27) or (Z28), which are optionally substituted. In the context of the present embodiment, Z preferably comprises a (hetero)cyclooctene moiety according to structure (Z40) or (Z41) as shown below, wherein Y¹ is O or NR¹¹, wherein R¹¹ is independently selected from the group consisting of hydrogen, a linear or branched C₁-C₁₂ alkyl group or a C₄ -C₁₂ (hetero)aryl group. The aromatic rings in (Z40) are optionally O-sulfonylated at one or more positions, whereas the rings of (Z41) may be halogenated at one or more positions. Most preferably, Z is according to structure (Z43), defined further below.

In an alternative preferred embodiment, Z comprises a heterocycloheptenyl group and is according to structure (Z37).

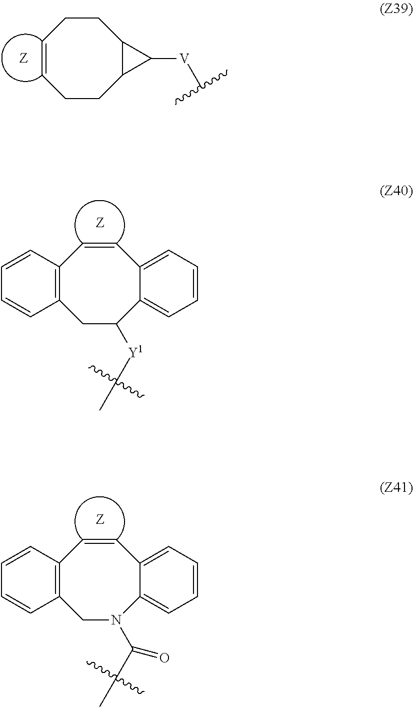

In an especially preferred embodiment, Z comprises a cyclooctenyl group and is according to structure (Z42):

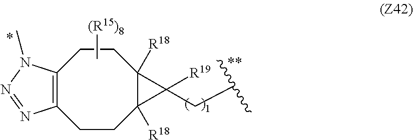

### Herein:

the bond labelled with * is connected to Su and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q¹ or D connected via a spacer moiety; and
I is an integer in the range 0 to 10.

In a preferred embodiment of the group according to structure (Z42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the group according to structure (Z42), R¹⁸ is independently selected from the group consisting of hydrogen, C₁-C₆ alkyl groups, most preferably both R¹⁸ are H. In a preferred embodiment of the group according to structure (Z42), R¹⁹ is H. In a preferred embodiment of the group according to structure (Z42), I is 0 or 1, more preferably I is 1.

In an especially preferred embodiment, Q¹ comprises a (hetero)cyclooctynyl group and is according to structure (Z43):

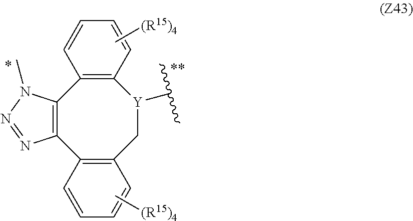

### Herein:

the bond labelled with * is connected to Su and the wavy bond labelled with ** is connected to L;
R¹⁵ is independently selected from the group consisting of hydrogen, halogen, —OR¹⁶, −NO₂, —CN, —S(O)₂R¹⁶, −S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the group according to structure (Z43), R¹⁵ is independently selected from the group consisting of hydrogen, halogen, −OR¹⁶, −S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and −S(O)₃⁽⁻⁾. In a preferred embodiment of the group according to structure (Z43), Y is N or CH, more preferably Y=N.

In an alternative preferred embodiment, Z comprises a (hetero)cycloalkane moiety, i.e. the bond depicted as [symbol] is a single bond. The (hetero)cycloalkane group may also be referred to as a heterocycloalkanyl group or a cycloalkanyl group, preferably a cycloalkanyl group, wherein the (hetero)cycloalkanyl group is optionally substituted. Preferably, the (hetero)cycloalkanyl group is a (hetero)cyclopropanyl group, a (hetero)cyclobutanyl group, a norbornane group, a norbornene group, a (hetero)cycloheptanyl group, a (hetero)cyclooctanyl group, a (hetero)cyclononnyl group or a (hetero)cyclodecanyl group, which may all optionally be substituted. Especially preferred are (hetero)cyclopropanyl groups, (hetero)cycloheptanyl group or (hetero)cyclooctanyl groups, wherein the (hetero)cyclopropanyl group, the trans-(hetero)cycloheptanyl group or the (hetero)cyclooctanyl group is optionally substituted. Preferably, Z comprises a cyclopropanyl moiety according to structure (Z44), a hetereocyclobutane moiety according to structure (Z45), a norbornane or norbornene group according to structure (Z46), a (hetero)cycloheptanyl moiety according to structure (Z47) or a (hetero)cyclooctanyl moiety according to structure (Z48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropanyl group is according to structure (Z49). In another preferred embodiment, the (hetero)cycloheptane group is according to structure (Z50) or (Z51). In another preferred embodiment, the (hetero)cyclooctane group is according to structure (Z52), (Z53), (Z54), (Z55) or (Z56).

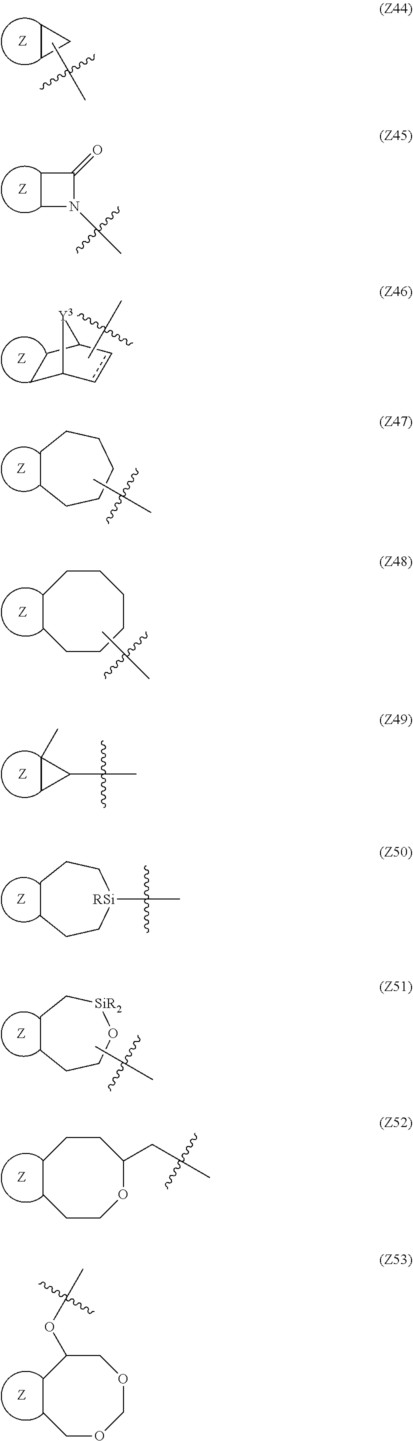

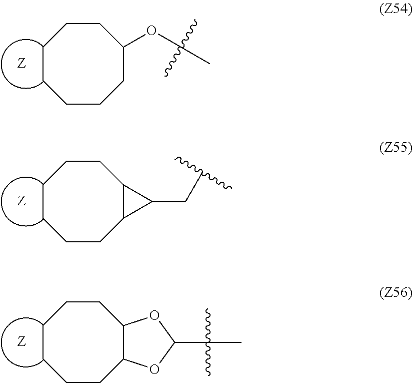 [SMILES: *OC1CCCCCCC1 ; *CC1C2CCCCCCC12 ; *C1OC2CCCCCCC2O1]

Herein, the R group(s) on Si in (Z50) and (Z51) are typically alkyl or aryl, preferably C₁-C₆ alkyl. Ring Z is selected from structures (Zk)-(Zn), wherein the carbon atoms labelled with ** correspond to the two carbon atoms of the (hetero)cycloalkane ring of (Z44)-(Z56) to which ring Z is fused, and the carbon a carbon labelled with * is directly connected to the peptide chain of the antibody. Since the connecting group Z is formed by reaction with a (hetero)cycloalkene in the context of the present embodiment, the bound depicted above as [symbol] is a single bond.

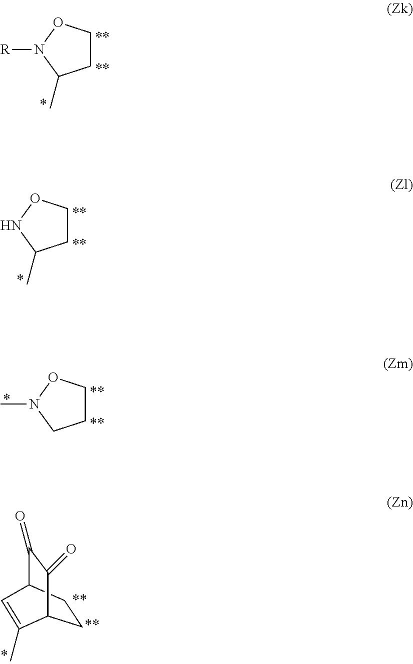

In a second preferred embodiment, Z is formed by a nucleophilic reaction, preferably by a nucleophilic substitution ora Michael addition, preferably by a Michael addition. A preferred Michael reaction is the thiol-maleimide ligation, most preferably wherein Q is maleimide and F is a thiol group. In a preferred embodiment, connection group Z comprises a succinimidyl ring or its ring-opened succinic acid amide derivative. Preferred options for connection group Z comprise a moiety selected from (Z57)-(Z66) depicted here below.

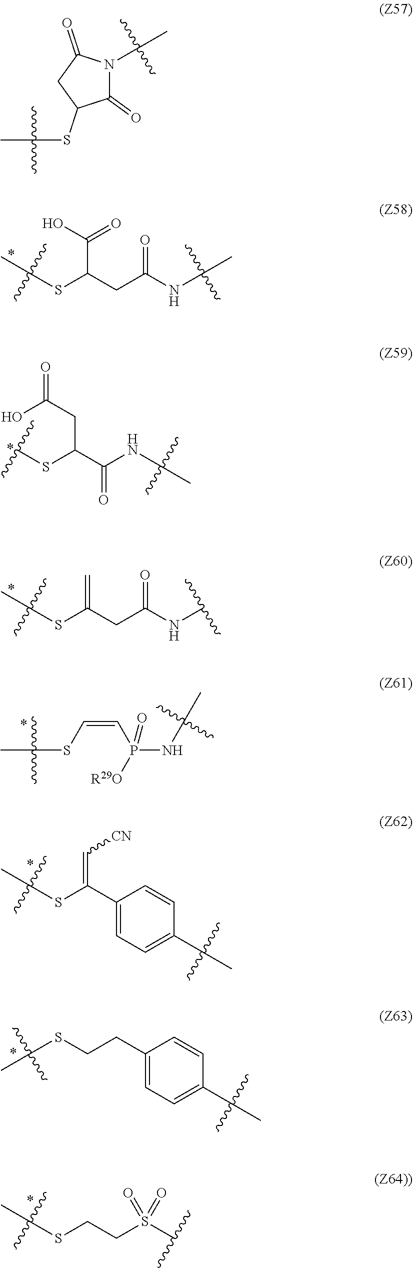

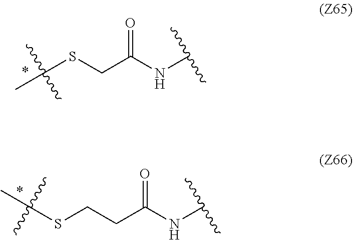 [SMILES: *NC(=O)CS* ; *NC(=O)CCS*]

Herein, the wavy bond(s) labelled with an * is connected to Su, and the other wavy bond to L. In addition, R²⁹ is C₁₋₁₂ alkyl, preferably C₁₋₄ alkyl, most preferably ethyl.

In a preferred embodiment, connection group Z comprise a moiety selected from (Z1)-(Z66).

### Linker L

Linkers, also referred to as linking units, are well known in the art and any suitable linker may be used. In the compound of structure (3), linker L connects chemical handle Q with payload D. In the conjugate of structure (1), linker L connects connecting group Z with payload D. The linker may be a cleavable or non-cleavable linker. The linker may contain one or more branch-points for attachment of multiple payloads D to a reactive moiety Q.

The linker may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR¹², wherein y is 0, 1 or 2, preferably y=2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. The linker may contain (poly)ethylene glycoldiamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z-diaminoalkanes wherein z is the number of carbon atoms in the alkane, and may for example range from 2-25.

In a preferred embodiment, linker L comprises a sulfamide group, preferably a sulfamide group according to structure (L1):

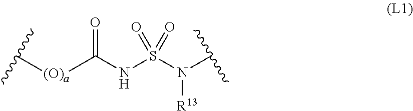

The wavy lines represent the connection to the remainder of the compound or conjugate, typically to Q or Z and to D, optionally via a spacer. Preferably, the (O)ₐC(O) moiety is connected to Q or Z and the NR¹³ moiety to D.

In structure (L1), a=0 or 1, preferably a=1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of Q² or D connected to N via a spacer moiety, preferably Sp² as defined here below.

In a preferred embodiment, R¹³ is hydrogen or a C₁-C₂₀ alkyl group, more preferably R¹³ is hydrogen or a C₁-C₁₆ alkyl group, even more preferably R¹³ is hydrogen or a C₁-C₁₀ alkyl group, wherein the alkyl group is optionally substituted and optionally interrupted by one or more heteroatoms selected from O, S and NR^{14,} preferably O, wherein R¹⁴ is independently selected from the group consisting of hydrogen and C₁-C₄ alkyl groups. In a preferred embodiment, R¹³ is hydrogen. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an −OH group, preferably a terminal −OH group. In this embodiment it is further preferred that R¹³ is a (poly)ethylene glycol chain comprising a terminal −OH group. In another preferred embodiment, R¹³ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R¹³ is hydrogen or methyl, and most preferably R¹³ is hydrogen.

In a preferred embodiment, the linker is according to structure (L2):

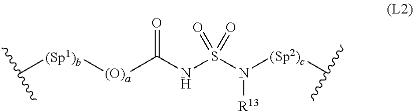

Herein, a, R¹³ and the wavy lines are as defined above, Sp¹ and Sp² are independently spacer moieties and b and c are independently 0 or 1. Preferably, b=0 or 1 and c=1, more preferably b=0 and c=1. In one embodiment, spacers Sp¹ and Sp²are independently selected from the groups consisting of linear or branched C₁-C₂₀₀ alkenylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkyene groups, cycloalkenylene groups, cylcoalkynylene groups, alkylarlylene groups, arylalkylene groups, arylalkenylene groups and arylalkynylene groups being optionally substituted and optionally interrupted by one or more heteroatoms selected from the group of O, S, and NR²⁰, wherein R²⁰ is independently selected from the groups consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cyloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S, and NR²⁰, wherein R²⁰ is independently selected from the group consisting of hydrogen, C₁-C₂₄ alkyl groups, C₂-C₂₄ alkenyl groups, C₂-C₂₄ alkynyl groups, C₃-C₂₄ cycloalkyl groups, the alkyl groups, alkenyl groups, alkynyl groups and cycloalkyl groups being optionally substituted.

Even more preferably, spacer moieties Sp¹ and Sp², if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cylcloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR²⁰, preferably O, wherein R²⁰ is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR²⁰, preferably O and/or S—S, wherein R²⁰ is independently selected from the group consisting of hydrogen and C₁-C₄ alkyl groups, preferably hydrogen or methyl.

Another class of suitable linkers comprises cleavable linkers. Cleavable linkers are well known in the art. For example Shabat et al., Soft Matter 2012, 6, 1073, incorporated by reference herein, discloses cleavable linkers comprising self-immolative moieties that are released upon a biological trigger, e.g. an enzymatic cleavage or an oxidation event. Some examples of suitable cleavable linkers are peptide-linkers that are cleaved upon specific recognition by a protease, e.g. cathepsin, plasmin or metalloproteases, or glycoside-based linkers that are cleaved upon specific recognition by a glycosidase, e.g. glucuronidase, or nitroaromatics that are reduced in oxygen-poor, hypoxic areas.

Linker L may further contain a peptide spacer as known in the art, preferably a dipeptide or tripeptide spacer as known in the art, preferably a dipeptide spacer. Although any dipeptide or tripeptide spacer may be used, preferably the peptide spacer is selected from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, more preferably Val-Cit, Val-Ala, Ala-Ala-Asn. In one embodiment, the peptide spacer is Val-Cit. In one embodiment, the peptide spacer is Val-Ala. The peptide spacer may also be attached to the payload, wherein the amino end of the peptide spacer is conveniently used as amine group in the method according to the first aspect of the invention.

In a preferred embodiment, the peptide spacer is represented by general structure (L3):

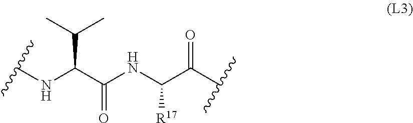

Herein, R¹⁷=CH₃ (Ala) or CH₂CH₂CH₂NHC(O)NH₂ (Cit). The wavy lines indicate the connection to the remainder of the molecule, preferably the peptide spacer according to structure (L3) is connected via NH to Q or Z, typically via a spacer, and via C(O) to D, typically via a spacer.

Linker L may further contain a self-cleavable spacer, also referred to as self-immolative spacer. The self-cleavable spacer may also be attached to the payload. Preferably, the self-cleavable spacer is para-aminobenzyloxycarbonyl (PABC) derivative, more preferably a PABC derivative according to structure (L4).

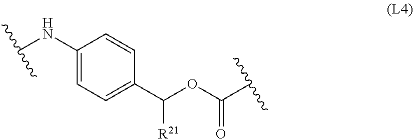

Herein, the wavy lines indicate the connection to the remainder of the molecule. Typically, the PABC derivative is connected via NH to Q or Z, typically via a spacer, and via OC(O) to D, typically via a spacer. Preferably, the PABC derivative (L4) is connected via NH directly to the C(O) of (L3).

R²¹ is H, R²² or C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups. Preferably, R²² is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R²¹ is H or C(O)R²², wherein R²²⁼⁴-methyl-piperazine or morpholine. Most preferably, R²¹ is H.

### Payload D

Linker L connects Z with payload D. Payload molecules are well-known in the art, especially in the field of antibody-drug conjugates, as the moiety that is covalently attached to the antibody and that is released therefrom upon uptake of the conjugate and/or cleavage of the linker. In a preferred embodiment, the payload is selected from the group consisting of an active substance, a reporter molecule, a polymer, a solid surface, a hydrogel, a nanoparticle, a microparticle and a biomolecule. Especially preferred payloads are active substances and reporter molecules, in particular active substances.

The term “active substance” herein relates to a pharmacological and/or biological substance, i.e. a substance that is biologically and/or pharmaceutically active, for example a drug, a prodrug, a cytotoxin, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine.

When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterial agents, peptides and oligonucleotides. Examples of cytotoxins include colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, auristatins, enediynes, pyrrolobenzodiazepines (PBDs) or indolinobenzodiazepine dimers (IGN) or PNU159,682 and derivatives thereof. Preferred payloads are selected from MMAE, MMAF, exatecan, SN-38, DXd, maytansinoids, calicheamicin, PNU159,685 and PBD dimers. Especially preferred payloads are PBD, SN-38, MMAE, exatecan or DXd. In one embodiment, the payload is MMAE. In one embodiment, the payload is exatecan or DXd. In one embodiment, the payload is SN-38. In one embodiment, the payload is MMAE. In one embodiment, the payload is a PDB dimer.

The term “reporter molecule” herein refers to a molecule whose presence is readily detected, for example a diagnostic agent, a dye, a fluorophore, a radioactive isotope label, a contrast agent, a magnetic resonance imaging agent or a mass label.

A wide variety of fluorophores, also referred to as fluorescent probes, is known to a person skilled in the art. Several fluorophores are described in more detail in e.g. G. T. Hermanson, “Bioconjugate Techniques”, Elsevier, 3^{rd} Ed. 2013, Chapter 10: “Fluorescent probes”, p. 395 - 463, incorporated by reference. Examples of a fluorophore include all kinds of Alexa Fluor (e.g. Alexa Fluor 555), cyanine dyes (e.g. Cy3 or Cy5) and cyanine dye derivatives, coumarin derivatives, fluorescein and fluorescein derivatives, rhodamine and rhodamine derivatives, boron dipyrromethene derivatives, pyrene derivatives, naphthalimide derivatives, phycobiliprotein derivatives (e.g. allophycocyanin), chromomycin, lanthanide chelates and quantum dot nanocrystals.

Examples of a radioactive isotope label include ^{99m}Tc, ¹¹¹In, ^{114m}In, ¹¹⁵In, ¹⁸F, ¹⁴C, ⁶⁴Cu, ¹³¹I, ¹²⁵I, ¹²³I, ²¹²Bi, ⁸⁸Y, ⁹⁰Y, ⁶⁷Cu, ¹⁸⁶Rh, ¹⁸⁸Rh, ⁶⁶Ga, ⁶⁷Ga and ¹⁰B, which is optionally connected via a chelating moiety such as e.g. DTPA (diethylenetriaminepentaacetic anhydride), DOTA (1,4,7,10-tetraazacyclododecane-N,N′,N″,N″′-tetraacetic acid), NOTA (1,4,7-triazacyclononane N,N′,N″-triacetic acid), TETA (1,4,8,11-tetraazacyclotetradecane-N,N′,N″,N″′-tetraacetic acid), DTTA (N¹-(p-isothiocyanatobenzyl)-diethylenetriamine-N¹,N²,N³,N³-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide). Isotopic labelling techniques are known to a person skilled in the art, and are described in more detail in e.g. G. T. Hermanson, “Bioconjugate Techniques”, Elsevier, 3^{rd} Ed. 2013, Chapter 12: “Isotopic labelling techniques”, p. 507-534, incorporated by reference.

Polymers suitable for use as a payload D in the compound according to the invention are known to a person skilled in the art, and several examples are described in more detail in e.g. G. T. Hermanson, “Bioconjugate Techniques”, Elsevier, 3^{rd} Ed. 2013, Chapter 18: “PEGylation and synthetic polymer modification”, p. 787-838, incorporated by reference. When payload D is a polymer, payload D is preferably independently selected from the group consisting of a poly(ethyleneglycol) (PEG), a polyethylene oxide (PEO), a polypropylene glycol (PPG), a polypropylene oxide (PPO), a 1,q-diaminoalkane polymer (wherein q is the number of carbon atoms in the alkane, and preferably q is an integer in the range of 2 to 200, preferably 2 to 10), a (poly)ethylene glycol diamine (e.g. 1,8-diamino-3,6-dioxaoctane and equivalents comprising longer ethylene glycol chains), a polysaccharide (e.g. dextran), a poly(amino acid) (e.g. a poly(L-lysine)) and a poly(vinyl alcohol).

Solid surfaces suitable for use as a payload D are known to a person skilled in the art. A solid surface is for example a functional surface (e.g. a surface of a nanomaterial, a carbon nanotube, a fullerene or a virus capsid), a metal surface (e.g. a titanium, gold, silver, copper, nickel, tin, rhodium or zinc surface), a metal alloy surface (wherein the alloy is from e.g. aluminum, bismuth, chromium, cobalt, copper, gallium, gold, indium, iron, lead, magnesium, mercury, nickel, potassium, plutonium, rhodium, scandium, silver, sodium, titanium, tin, uranium, zinc and/or zirconium), a polymer surface (wherein the polymer is e.g. polystyrene, polyvinylchloride, polyethylene, polypropylene, poly(dimethylsiloxane) or polymethylmethacrylate, polyacrylamide), a glass surface, a silicone surface, a chromatography support surface (wherein the chromatography support is e.g. a silica support, an agarose support, a cellulose support or an alumina support), etc. When payload D is a solid surface, it is preferred that D is independently selected from the group consisting of a functional surface or a polymer surface.

Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. When the payload is a hydrogel, it is preferred that the hydrogel is composed of poly(ethylene)glycol (PEG) as the polymeric basis.

Micro- and nanoparticles suitable for use as a payload D are known to a person skilled in the art. A variety of suitable micro- and nanoparticles is described in e.g. G. T. Hermanson, “Bioconjugate Techniques”, Elsevier, 3^{rd} Ed. 2013, Chapter 14: “Microparticles and nanoparticles”, p. 549-587, incorporated by reference. The micro- or nanoparticles may be of any shape, e.g. spheres, rods, tubes, cubes, triangles and cones. Preferably, the micro- or nanoparticles are of a spherical shape. The chemical composition of the micro- and nanoparticles may vary. When payload D is a micro- or a nanoparticle, the micro- or nanoparticle is for example a polymeric micro- or nanoparticle, a silica micro- or nanoparticle or a gold micro- or nanoparticle. When the particle is a polymeric micro- or nanoparticle, the polymer is preferably polystyrene or a copolymer of styrene (e.g. a copolymer of styrene and divinylbenzene, butadiene, acrylate and/or vinyltoluene), polymethylmethacrylate (PMMA), polyvinyltoluene, poly(hydroxyethyl methacrylate (pHEMA) or poly(ethylene glycol dimethacrylate/2-hydroxyethylmetacrylae) [poly(EDGMA/HEMA)]. Optionally, the surface of the micro- or nanoparticles is modified, e.g. with detergents, by graft polymerization of secondary polymers or by covalent attachment of another polymer or of spacer moieties, etc.

Payload D may also be a biomolecule. Biomolecules, and preferred embodiments thereof, are described in more detail below. When payload D is a biomolecule, it is preferred that the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides.

In the context of the present invention, cytotoxic payloads are especially preferred. Thus, D is preferably, a cytotoxin, more preferably selected from the group consisting of colchicine, vinca alkaloids, anthracyclines, camptothecins, doxorubicin, daunorubicin, taxanes, calicheamycins, tubulysins, irinotecans, an inhibitory peptide, amanitins, amatoxins, deBouganin, duocarmycins, epothilones, mytomycins, combretastatins, maytansines, auristatins, enediynes, pyrrolobenzodiazepines (PBDs) or indolinobenzodiazepine dimers (IGN) or PNU159,682. In an especially preferred embodiment, D is MMAE or exatecan.

### Method for Preparing an Antibody Conjugate

The invention further concerns in a third aspect a method for preparing the antibody conjugate according to the invention. The method comprises the following steps:
(a) expressing an antibody in a mammalian expression system, optionally in the presence of a glycocidase or a glycosyltransferase inhibitor;
(b) optionally subjecting the expressed antibody to deglycosylation with an enzyme selected from an alpha-mannosidase, galactosidase and sialidase;
(c) contacting the optionally deglycosylated antibody with a saccharide moiety of structure Nuc-Su(F)ₓ in the presence of a glycosyltransferase to obtain a modified antibody having structure (2):

Ab-[(GlcNAc(Fuc)_{b}-(G)ₑ-(Su-(F)ₓ)ₛ]_{y} (2)
wherein Ab, GlcNAc, Fuc, G, Su, b, e, s, x and y are as defined above, Nuc is a nucleotide and F is reactive moiety capable of reacting in a cycloaddition or a nucleophilic reaction;
(d) conjugating the modified antibody having structure (2) with a linker payload construct having structure (3):

Q-L-(D)ᵣ (3)
wherein L, D and r are as defined above and Q is reactive moiety capable of reacting with F in a cycloaddition or a nucleophilic reaction, to obtain an antibody conjugate having structure (1):

Ab-[(GlcNAc(Fuc)_{b}-(G)ₑ-(Su-(Z-L-(D)ᵣ)ₓ)ₛ]_{y} (1)
wherein Ab, GlcNAc, Fuc, G, Su, b, e, s, x, y, L, D and r are as defined above and Z is a connecting group formed by the reaction of F with Q in a cycloaddition or a nucleophilic reaction, and is further defined above.

### Step (a)—Expression

Expression of antibodies is well-known in the art. In a preferred embodiment, a FUT8 knock-out expression system is used. In such an expression system, the antibodies formed are not fucosylated, i.e. b=0.

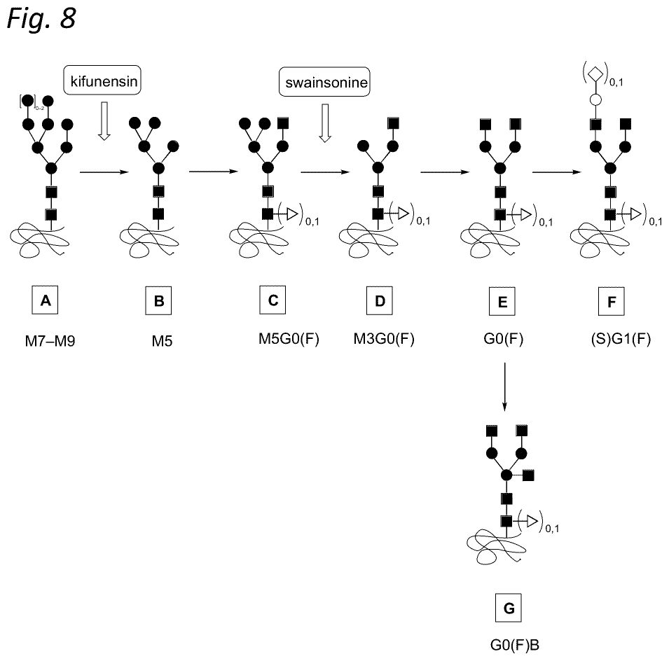

In a further preferred embodiment, expression is done in the presence of a glycosidase or a glycosyltransferase inhibitor, preferably a glycosyltransferase inhibitor. The glycosyltransferase inhibitor may be a fucosyltransferase inhibitor, a galactosyltransferase inhibitor, a sialyltransferase inhibitor or a mannosidase inhibitor. In a preferred embodiment, the glycosyltransferase inhibitor is a mannosidase inhibitor, most preferably swainsonine or kifunensin. As such, modified glycans having fewer mannose residues are formed, which are ideally suited for preparing the preferred antibody conjugates according to the invention. The thus obtained glycans are depicted in FIG. 8. In another preferred embodiment, the glycosyltransferase inhibitor is a fucosyltransferase inhibitor, such as fucostatin I, fucostatin II, 2-fluorofucose, 6-fluorinated derivative of fucose, Fucotrim I or Fucotrim II, or acylated variants thereof. As such, modified glycans having fewer or no fucose residues are formed, which are ideally suited for preparing the preferred antibody conjugates according to the invention.

### Step (b)—Deglycosylation

The expressed antibody may be subjected to deglycosylation, but this is not always necessary and depends on the structure of the glycan formed in step (a). In case deglycosylation occurs, it is typically performed with an enzyme selected from an alpha-mannosidase, a galactosidase and a sialidase. No deglycosylation with an endoglycosidase or an amidase is performed in the method according to this aspect, as such enzymes would remove a too large part of the glycan, giving antibodies with e below 4.

In a preferred embodiment, the deglycosylation of step (b) is performed, preferably with an alpha-mannosidase, a galactosidase and/or a sialidase. Herein, the alpha-mannosidase may be selected from alpha-mannosidase I and alpha-mannosidase II, preferably alpha-mannosidase I.

### Step (c)—Glycosyltransfer

The optionally deglycosylated antibody is subjected to glycosyltransfer in order to attach Su(F)ₓ to (G)ₑ. The antibody is contacted with a saccharide moiety of structure Nuc-Su(F)ₓ (nucleotide sugar) in the presence of a glycosyltransferase enzyme, to obtain a modified antibody having structure (2):

Ab-[(GlcNAc(Fuc)_{b}-(G)ₑ-(Su-(F)ₓ)ₛ]_{y} (2)

Herein, Ab, GlcNAc, Fuc, G, Su, b, e, s, x and y are as defined above, Nuc is a nucleotide and F is reactive moiety capable of reacting in a cycloaddition or a nucleophilic reaction. F is further defined below. Nuc is preferably GDP, CMP or UDP.

Glycosyltransfer using a glycosyltransferase enzyme is well-known in the art, and may be performed by any suitable glycosyltransferase enzyme, such as MGAT-I, MGAT-III, MGAT-IV, MGAT-V, galactosyltransferase (GalT), N-acetylgalactosylaminetransferase (GalNAcT) and sialyltransferase (SialT). The skilled person is able to match the desired nucleotide sugar with the desired glycosyltransferase.

### Step (d)—Conjugation

The modified antibody having structure (2) is conjugated to a linker payload construct having structure (3):

Q-L-(D)ᵣ (3)

Herein, L, D and r are as defined above and Q is reactive moiety capable of reacting with F in a cycloaddition or a nucleophilic reaction. Q is further defined below. Such conjugation reactions are well-known to the skilled person, for example from Hermanson, “Bioconjugate Techniques”, Elsevier, 3^{rd} Ed. 2013, and WO 2014/065661, both incorporated by reference.

### Reactive Moiety Q

Q serves as chemical handle for the connection to Su(F)ₓ. In other words, Q is reactive towards F in a cycloaddition or a nucleophilic reaction. Preferably, Q comprises a click probe, a thiol or a thiol-reactive moiety. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, the click probe comprises or is an alkene moiety or an alkyne moiety, more preferably wherein the alkene is a (hetero)cycloalkene and/or the alkyne is a terminal alkyne or a (hetero)cycloalkyne. Typical thiol-reactive moieties are selected from maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety. Most preferably, the thiol-reactive moiety comprises or is a maleimide moiety. In a preferred embodiment, Q is selected from an alkene moiety, an alkyne moiety, or a thiol-reactive moiety, more preferably an alkene moiety or an alkyne moiety, even more preferably an alkyne moiety. Herein, the alkene is preferably a (hetero)cycloalkene and the alkyne is preferably a terminal alkyne or a (hetero)cycloalkyne. Most preferably, Q is a cyclic (hetero)alkyne moiety. Each of these moieties are further defined here below.

Thus, in an especially preferred embodiment, Q comprises a cyclic (hetero)alkyne moiety. The alkynyl group may also be referred to as a (hetero)cycloalkynyl group, i.e. a heterocycloalkynyl group or a cycloalkynyl group, wherein the (hetero)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Herein, the (hetero)cycloalkynes may optionally be substituted. Preferably, the (hetero)cycloalkynyl group is an optionally substituted (hetero)cycloheptynyl group or an optionally substituted (hetero)cyclooctynyl group. Most preferably, the (hetero)cycloalkynyl group is a (hetero)cyclooctynyl group, wherein the (hetero)cyclooctynyl group is optionally substituted.

In an especially preferred embodiment, Q comprises an (hetero)cycloalkynyl group and is according to structure (Q1):

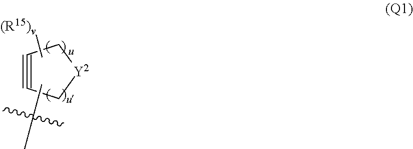

Herein:
R¹⁵ is independently selected from the group consisting of hydrogen, halogen, −OR¹⁶, —NO2, −CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S⁽⁺⁾ is a cationic sulphur atom counterbalanced by B⁽⁻⁾, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5. Typically, v=(u+u′)×2 or [(u+u′)×2]−1. In a preferred embodiment, v=8, 9 or 10, more preferably v=9 or 10, most preferably v=10.

In a preferred embodiment, Q is selected from the group consisting of (Q2)—(Q20) depicted here below.

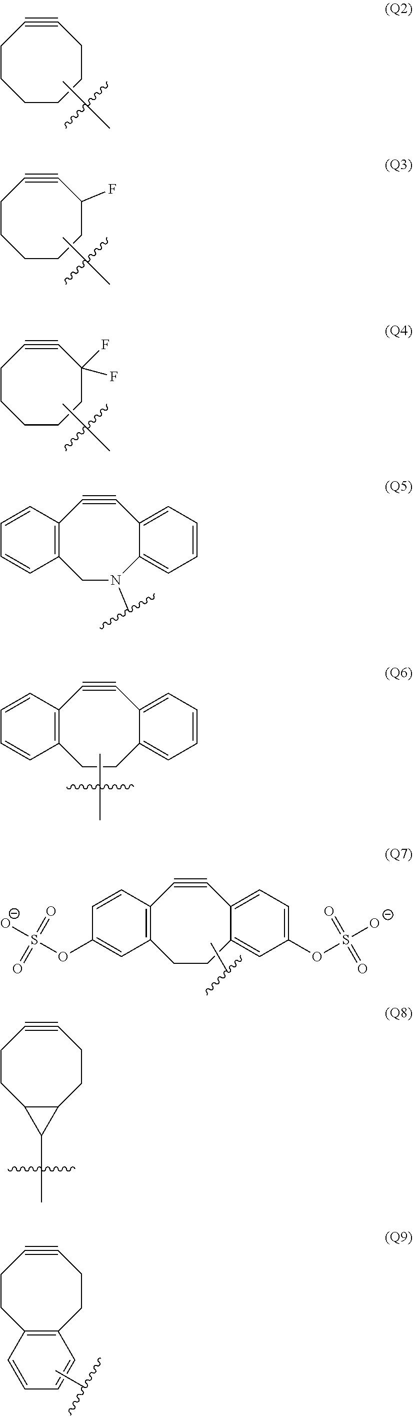

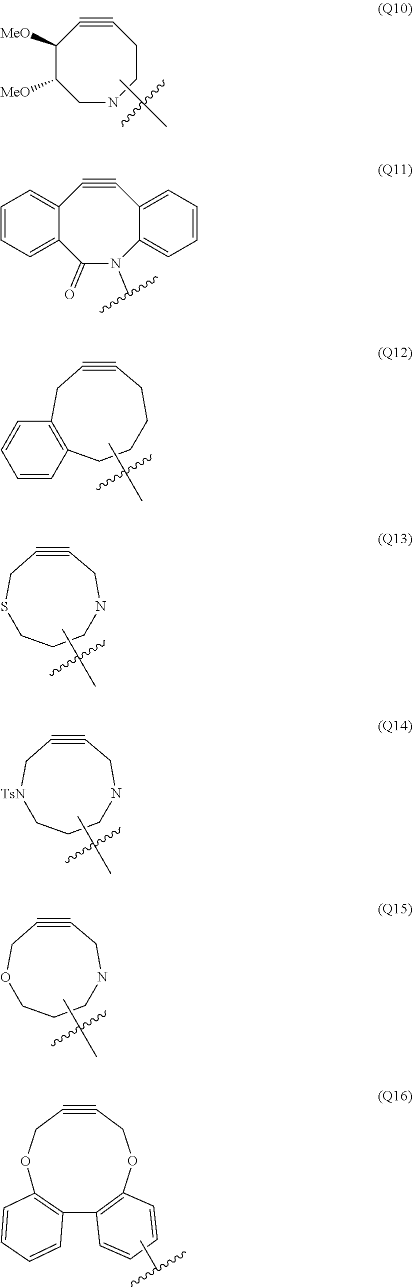

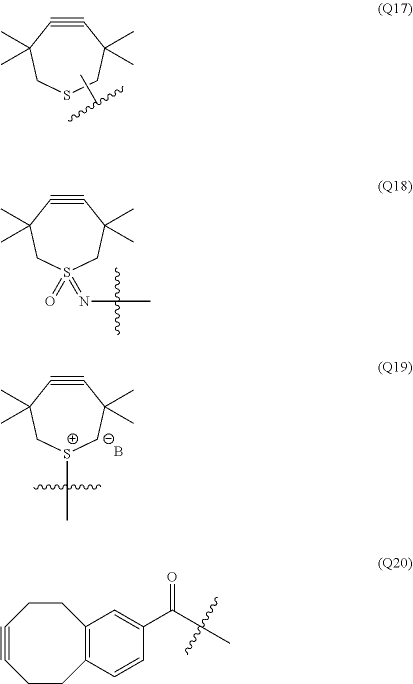

Herein, the connection to L, depicted with the wavy bond, may be to any available carbon or nitrogen atom of Q. The nitrogen atom of (Q10), (Q13), (Q14) and (Q15) may bear the connection to L, or may contain a hydrogen atom or be optionally functionalized. B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf. In the conjugation reaction, B⁽⁻⁾ does not need to be a pharmaceutically acceptable anion, since B⁽⁻⁾ will exchange with the anions present in the reaction mixture anyway. In case (Q19) is used for Q, the negatively charged counter-ion is preferably pharmaceutically acceptable upon isolation of the conjugate according to the invention, such that the conjugate is readily useable as medicament.

In a further preferred embodiment, Q is selected from the group consisting of (Q21)-(Q38) depicted here below.

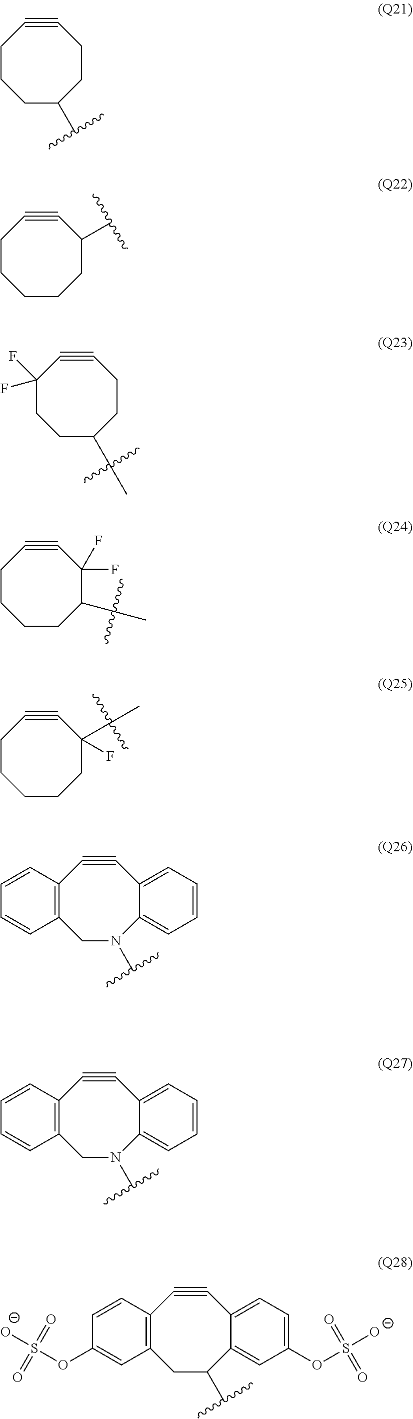

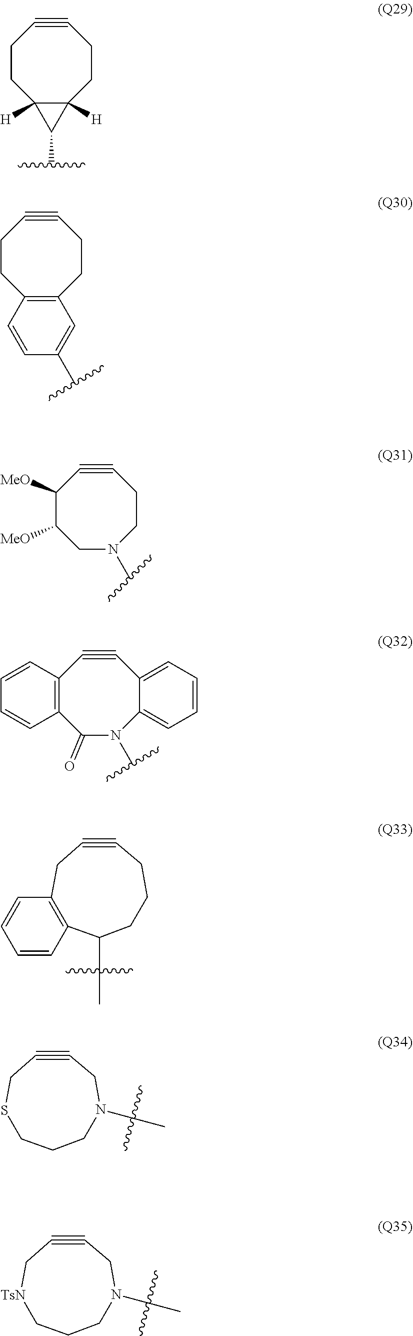

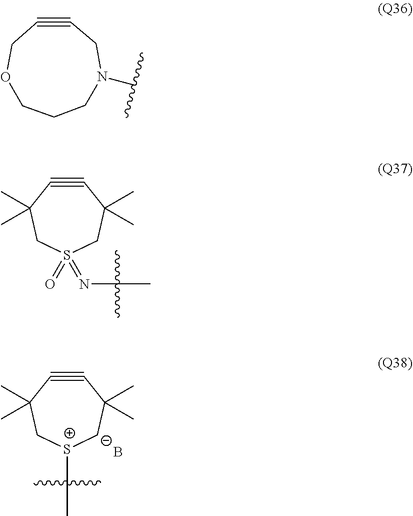

In structure (Q38), B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf.

In a preferred embodiment, Q comprises a (hetero)cyclooctyne moiety according to structure (Q8), more preferably according to (Q29), also referred to as a bicyclo[6.1.0]non-4-yn-9-yl] group (BCN group), which is optionally substituted. In the context of the present embodiment, Q preferably is a (hetero)cyclooctyne moiety according to structure (Q39) as shown below, wherein V is (CH₂)_{I} and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Q39), I is most preferably 1. Most preferably, Q is according to structure (Q42), defined further below.

In an alternative preferred embodiment, Q comprises a (hetero)cyclooctyne moiety according to structure (Q26), (Q27) or (Q28), also referred to as a DIBO, DIBAC, DBCO or ADIBO group, which are optionally substituted. In the context of the present embodiment, Q preferably is a (hetero)cyclooctyne moiety according to structure (Q40) or (Q41) as shown below, wherein Y¹ is O or NR¹¹, wherein R¹¹ is independently selected from the group consisting of hydrogen, a linear or branched C₁-C₁₂ alkyl group or a C₄ -C₁₂ (hetero)aryl group. The aromatic rings in (Q40) are optionally O-sulfonylated at one or more positions, whereas the rings of (Q41) may be halogenated at one or more positions. Most preferably, Q is according to structure (Q43), defined further below.

In an alternative preferred embodiment, Q comprises a heterocycloheptynyl group and is according to structure (Q37).

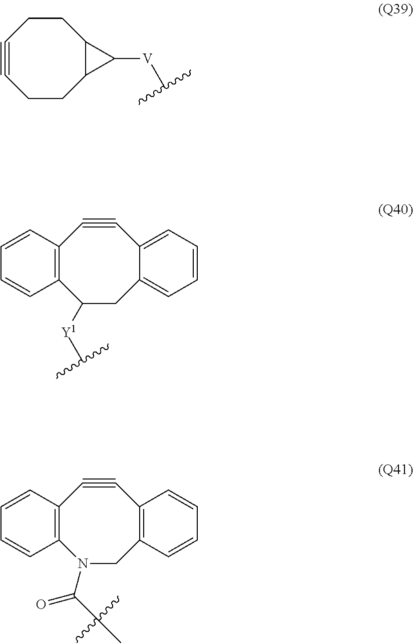

In an especially preferred embodiment, Q comprises a cyclooctynyl group and is according to structure (Q42):

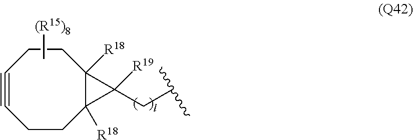

Herein:
R¹⁵ is independently selected from the group consisting of hydrogen, halogen, −OR¹⁶, —NO2, −CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q¹ or D connected via a spacer moiety; and
I is an integer in the range 0 to 10.

In a preferred embodiment of the reactive group according to structure (Q42), R¹⁵ is independently selected from the group consisting of hydrogen, halogen, −OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Q42), R¹⁸ is independently selected from the group consisting of hydrogen, C₁-C₆ alkyl groups, most preferably both R¹⁸ are H. In a preferred embodiment of the reactive group according to structure (Q42), R¹⁹ is H. In a preferred embodiment of the reactive group according to structure (Q42), I is 0 or 1, more preferably I is 1.

In an especially preferred embodiment, Q comprises a (hetero)cyclooctynyl group and is according to structure (Q43):

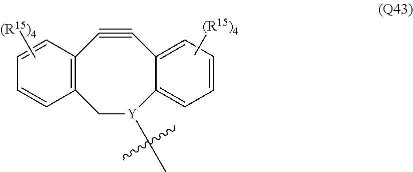

Herein:
R¹⁵ is independently selected from the group consisting of hydrogen, halogen, −OR¹⁶, —NO2, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Q43), R¹⁵ is independently selected from the group consisting of hydrogen, halogen, −OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (Q43), Y is N or CH, more preferably Y=N.

In an alternative preferred embodiment, Q comprises a cyclic alkene moiety. The alkenyl group Q may also be referred to as a (hetero)cycloalkenyl group, i.e. a heterocycloalkenyl group or a cycloalkenyl group, preferably a cycloalkenyl group, wherein the (hetero)cycloalkenyl group is optionally substituted. Preferably, the (hetero)cycloalkenyl group is a (hetero)cyclopropenyl group, a (hetero)cyclobutenyl group, a norbornene group, a norbornadiene group, a trans-(hetero)cycloheptenyl group, a trans-(hetero)cyclooctenyl group, a trans-(hetero)cyclononenyl group or a trans-(hetero)cyclodecenyl group, which may all optionally be substituted. Especially preferred are (hetero)cyclopropenyl groups, trans-(hetero)cycloheptenyl group or trans-(hetero)cyclooctenyl groups, wherein the (hetero)cyclopropenyl group, the trans-(hetero)cycloheptenyl group or the trans-(hetero)cyclooctenyl group is optionally substituted. Preferably, Q1 comprises a cyclopropenyl moiety according to structure (Q44), a hetereocyclobutene moiety according to structure (Q45), a norbornene or norbornadiene group according to structure (Q46), a trans-(hetero)cycloheptenyl moiety according to structure (Q47) or a trans-(hetero)cyclooctenyl moiety according to structure (Q48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropenyl group is according to structure (Q49). In another preferred embodiment, the trans-(hetero)cycloheptene group is according to structure (Q50) or (Q51). In another preferred embodiment, the trans-(hetero)cyclooctene group is according to structure (Q52), (Q53), (Q54), (Q55) or (Q56).

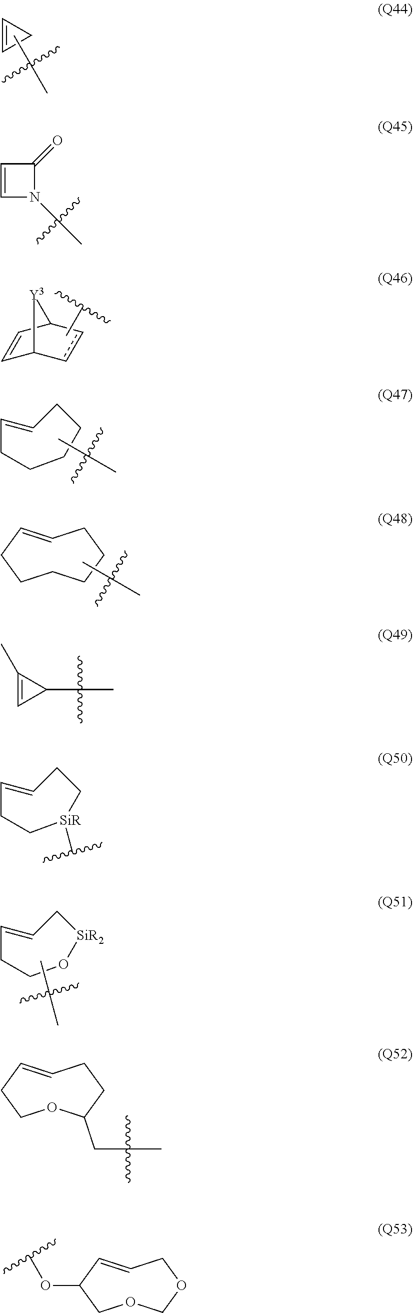

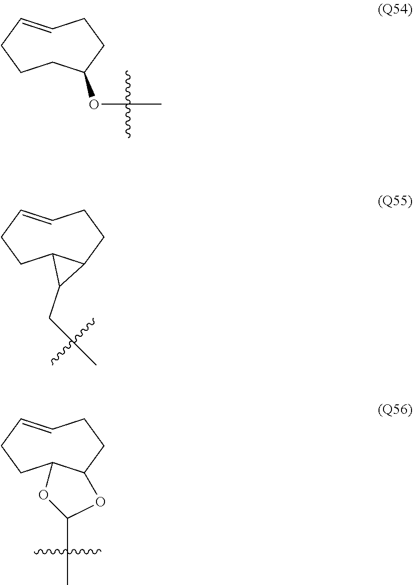

Herein, the R group(s) on Si in (Q50) and (Q51) are typically alkyl or aryl, preferably C₁-C₆ alkyl.

In an alternative preferred embodiment, Q is a thiol-reactive probe. Such probes are known in the art and may be selected from the group consisting of a maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety. Most preferably, Q comprises or is a maleimide moiety.

In a further preferred embodiment, probe Q is selected from the group consisting of (Q57)-(Q71) depicted here below.

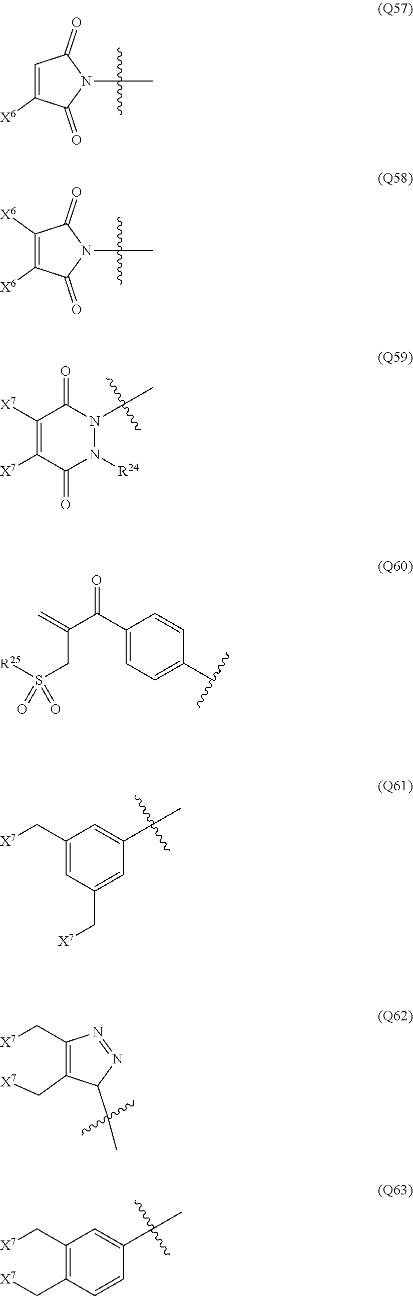

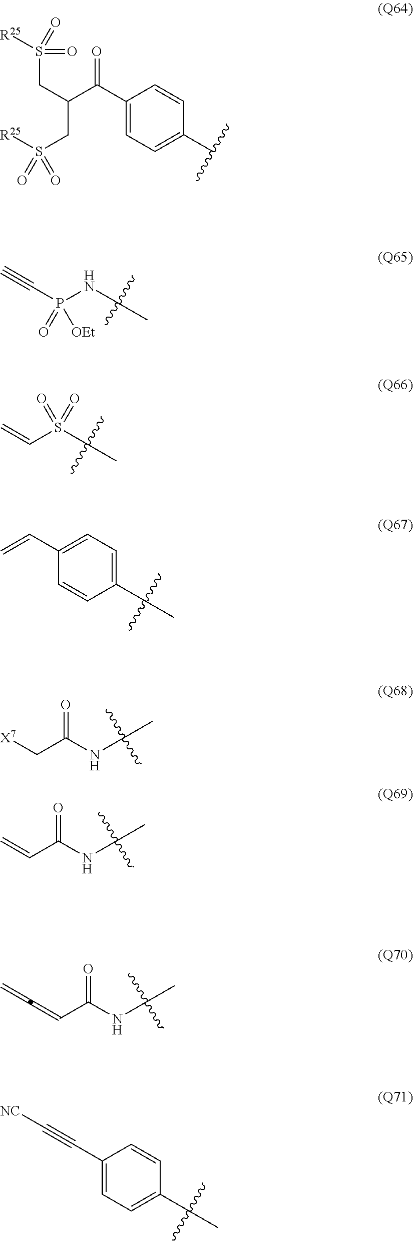

wherein:
X⁶ is H, halogen, PhS, MeS, preferably a halogen, such as Cl, Br, I;
X⁷ is halogen, PhS, MeS, preferably a halogen, such as Cl, Br, I;
R²⁴ is H or C₁₋₁₂ alkyl, preferably H or C₁₋₆ alkyl;
R²⁵ is H, C₁₋₁₂ alkyl, C₁₋₁₂ aryl, C₁₋₁₂ alkaryl or C₁₋₁₂ aralkyl, preferably H or para-methylphenyl;
wherein the aromatic ring of (Q61) and (Q63) may optionally be a heteroaromatic ring, such as a phenyl or pyridine ring.

In a preferred embodiment of thiol-reactive probe (Q57), the probe Q is selected from the group consisting of (Q72)—(Q74) depicted here below.

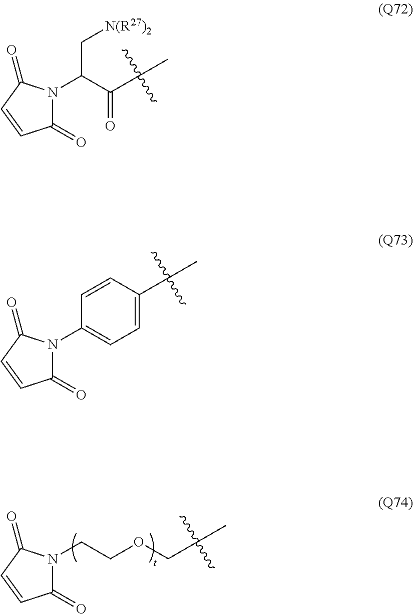

wherein:
R²⁷ is C₁₋₁₂ alkyl, C₁₋₁₂ aryl, C₁₋₁₂ alkaryl or C₁₋₁₂ aralkyl;
t is an integer in the range of 0-15, preferably 1-10.

In a preferred embodiment, Q is selected from the group consisting of (Q1)-(Q74).

### Reactive moiety F

F is reactive towards Q in a cycloaddition or a nucleophilic reaction. As the skilled person will understand, the options for F are the same as those for Q, provided that F and Q are reactive towards each other. Thus, F preferably comprises a click probe, a thiol or a thiol-reactive moiety. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, the click probe comprises or is an azide, a tetrazine, a triazine, a nitrone, a nitrile oxide, a nitrile imine, a diazo compound, an ortho-quinone, a dioxothiophene or a sydnone, most preferably an azide. Typical thiol-reactive moieties are selected from maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety. Most preferably, the thiol-reactive moiety comprises or is a maleimide moiety. In a preferred embodiment, F is a click probe or a thiol, more preferably F is an azide or a thiol, most preferably F is an azide.

Preferably, F is a click probe reactive towards a (hetero)cycloalkene and/or a (hetero)cycloalkyne, and is typically selected from the group consisting of azide, tetrazine, triazine, nitrone, nitrile oxide, nitrile imine, diazo compound, ortho-quinone, dioxothiophene and sydnone. Preferred structures for the reactive group are structures (F1)-(F10) depicted here below.

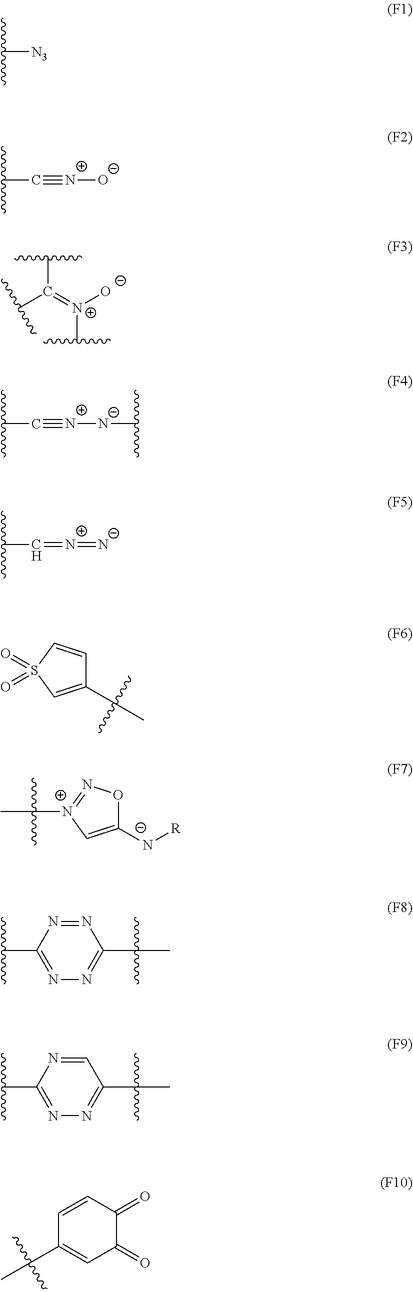

Herein, the wavy bond represents the connection to the payload. For (F3), (F4), (F8) and (F9), the payload can be connected to any one of the wavy bonds. The other wavy bond may then be connected to an R group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₃-C₂₄ sulfonyl groups, each of which (except hydrogen) may optionally be substituted and optionally interrupted by one or more heteroatoms selected from O, S and NR³² wherein R³² is independently selected from the group consisting of hydrogen and C₁-C₄ alkyl groups. The skilled person understands which R groups may be applied for each of the groups F. For example, the R group connected to the nitrogen atom of (F3) may be selected from alkyl and aryl, and the R group connected to the carbon atom of (F3) may be selected from hydrogen, alkyl, aryl, acyl and sulfonyl. Preferably, the click probe is selected from azides or tetrazines. Most preferably, the click probe is an azide.

### Application

The antibody conjugates according to the present invention are especially suitable in the treatment of cancer, by combining the mode-of-action of the cytotoxic payload with the effector function induced by opsonization of the cancer cell followed by recruitment and activation of immune cells. The invention thus further concerns the use of the antibody conjugates according to the present invention in medicine, preferably in the treatment of cancer. In a further aspect, the invention also concerns a method of treating a subject in need thereof, comprising administering the antibody conjugate according to the present invention to the subject. The method according to this aspect can also be worded as the antibody conjugate according to the present invention for use in treatment, in particular for use in the treatment of a subject in need thereof. The method according to this aspect can also be worded as use of the antibody conjugate according to the present invention for the manufacture of a medicament. Herein, administration typically occurs with a therapeutically effective amount of the antibody conjugate according to the present invention.

The invention further concerns a method for the treatment of a specific disease in a subject in need thereof, comprising the administration of the antibody conjugate according to the present invention as defined above. Typically, the specific disease is cancer and the subject in need thereof is a cancer patient. The use of antibody-drug conjugates is well-known in cancer treatment, and the antibody conjugate according to the present invention are especially suited in this respect. In the method according to this aspect, the conjugate is typically administered in a therapeutically effective amount. The present aspect of the invention can also be worded as the antibody conjugate according to the present invention for use in the treatment of a specific disease in a subject in need thereof, preferably for the treatment of cancer. In other words, this aspect concerns the use of the antibody conjugate according to the present invention for the preparation of a medicament or pharmaceutical composition for use in the treatment of a specific disease in a subject in need thereof, preferably for use in the treatment of cancer.

Administration in the context of the present invention refers to systemic administration. Hence, in one embodiment, the methods defined herein are for systemic administration of the conjugate. In view of the specificity of the conjugates, they can be systemically administered, and yet exert their activity in or near the tissue of interest (e.g. a tumour). Systemic administration has a great advantage over local administration, as the drug may also reach tumour metastasis not detectable with imaging techniques and it may be applicable to hematological tumours.

The invention further concerns a pharmaceutical composition comprising the antibody conjugate according to the present invention and a pharmaceutically acceptable carrier.

### EXAMPLES

The invention is illustrated by the following examples.

### General Reagents and Analytics

Solvents were purchased from Sigma-Aldrich or Fisher Scientific and used as received. Thin layer chromatography was performed on silica gel-coated plates (Kieselgel 60 F254, Merck, Germany) with the indicated solvent mixture, spots were detected by KMnO4 staining (1.5 g KMnO₄, 10 g K₂CO₃, 2.5 mL 5% NaOH-solution, 150 mL H₂O), p-anisaldehyde staining (9.2 mL p-anisaldehyde, 321 mL EtOH, 17 mL H2O, 3.75 mL AcOH, 12.7 mL H₂SO₄), and UV-detection. NMR spectra were recorded on a Bruker Biospin 400 (400 MHz) and a Bruker DMX300 (300 MHz). Protein mass spectra (HRMS) were recorded on a JEOL AccuTOF JMS-T100CS (Electrospray Ionization (ESI) time-of-flight) or a JEOL AccuTOF JMS-100GCv (Electron Ionization (EI), Chemical Ionization (CI)). Low-resolution mass spectra (LRMS) were recorded on a ThermoScientific Advantage LCQ Linear ion-trap electrospray and a Waters LCMS consisting of a 2767 Sample manager, 2525 pump, 2996 UV-detector and a Micromass ZQ with an Xbridge™ C18 3.5 μm column (ESI).

Trastuzumab (Herzuma or Ogivri) and cetuximab (Cerbitux) were obtained from the pharmacy.

### General Procedure for Reducing SDS-PAGE, Coomassie Staining and Fluorescence Detection

12% acrylamide gels were prepared according to BIO-RAD bulletin 6201 protocol. 5 μL 1 mg/mL antibody solution was diluted with 5 μL 2× sample buffer including 5% 2-mercaptoethanol and heated to 95° C. for 5 minutes. After loading the samples, the gel was run using a BIO-RAD Mini-PROTEAN Tetra Vertical Electrophoresis Cell at 150 volts until completion.

Fluorescently labelled proteins were analysed prior to staining using a BioRad ChemiDoc™ system. Subsequently, the gel was stained using staining solution, containing 1 g/L Coomassie Brilliant Blue R-250 in 5:4:1 (v/v/v) methanol:water:acetic acid, for 30 minutes. The gel was subsequently destained using 5:4:1 (v/v/v) methanol:water:acetic acid for 60 minutes, after which it was further destained overnight using demineralized water.

### General Procedure for Generation of Fc/2 Fragments

A solution of 20 μg of (modified) IgG was incubated for 1 hour at 37° C. with IdeS/Fabricator™ (1.25 U/μL) in PBS pH 6.6 in a total volume of 10 μL.

### General Procedure for Analytical RP-HPLC

Prior to RP-HPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-HPLC analysis was performed on an Agilent 1100 series (Hewlett Packard). The sample (10 μL) was injected with 0.5 mL/min onto Bioresolve RP mAb 2.1*150 mm 2.7 μm (Waters) with a column temperature of 70° C. A linear gradient was applied in 16.8 minutes from 30 to 54% acetonitrile in 0.1% TFA and water.

### General Procedure for Analytical SEC

HPLC-SEC analysis was performed on an Agilent 1100 series (Hewlett Packard) using an Xbridge BEH200A (3.5 μM, 7.8×300 mm, PN 186007640 Waters) column. The sample was diluted to 1 mg/mL in PBS and measured with 0.86 mL/min isocratic method (0.1 M sodium phosphate buffer pH 6.9 (NaHPO₄/Na₂PO₄) containing 10% isopropanol) for 16 minutes.

### General Procedure for Analytical MS Analysis

Prior to mass spectral analysis, IgG was treated with IdeS/Fabricator™, which allows analysis of the Fc/2 fragment. For analysis of the Fc/2 fragment, a solution of 20 μg (modified) IgG was incubated for 1 hour at 37° C. with IdeS/Fabricator™ (1.25 U/μL) in PBS pH 6.6 in a total volume of 10 μL. Samples were diluted to 80 μL followed by analysis electrospray ionization time-of-flight (ESI-TOF) on a JEOL AccuTOF. Deconvoluted spectra were obtained using Magtran software.

### Example 1. Enzymatic Remodeling of Trastuzumab to Trastuzumab-(G1F-6-azido-GalNAc)₂

Trastuzumab (7 mg, 23 mg/mL, obtained from the pharmacy) was incubated with TnGalNAcT (15% w/w), UDP 6-azidoGalNAc (75 eq compared to IgG), as described in WO2016170186, incorporated by reference, and calf intestine alkaline phosphatase (0.01% w/w, Roche) in 10 mM MnCl₂ and 20 mM tricine buffer pH 8.0 for 16 hours at 30° C. The functionalized IgG was purified using a protA column (25 mL, CaptivA PriMAB). After loading of the reaction mixture, the column was washed with TBS+0.2% triton and TBS. The IgG was eluted with 0.1 M NaOAc pH 2.7 and neutralized with 2.5 M Tris-HCl pH 7.2. After three times dialysis to PBS the functionalized trastuzumab was concentrated using a Vivaspin Turbo 4 ultrafiltration unit (Sartorius). Mass spectral analysis of a sample after IdeS treatment showed one major Fc/2 product (observed mass 25623 Da, approximately 50% of total Fc/2) corresponding to G1F with 1×6-azidoGalNAc and two minor products (observed mass 25689 Da, approximately 35% of total Fc/2) for G1F with 2×6-azidoGalNAc and (observed mass 25461Da, approximately 15% of total Fc/2) for G0F with 1×6-azidoGalNAc.

### Example 1b. Enzymatic Remodeling of Trastuzumab to Trastuzumab-(G1F-GalNAz)₂

Trastuzumab (50 mg, 15 mg/mL, obtained from the pharmacy) was incubated with TnGalNAcT (2% w/w), UDP-GalNAz (5 eq compared to IgG), as described in WO2016170186, incorporated by reference, and calf intestine alkaline phosphatase (0.01% w/w, Roche) in 6 mM MnCl₂ and 20 mM tricine buffer pH 8.0 for 16 hours at 30° C. The functionalized IgG was purified using a protA column (5 mL, MabSelect™ Sure™, Cytiva, as described in example 1). Subsequently the solution was dialyzed to TBS and the functionalized trastuzumab was concentrated using a Vivaspin Turbo 4 ultrafiltration unit (Sartorius). Mass spectral analysis of a sample after IdeS treatment showed one major Fc/2 product (observed mass 25717 Da, approximately 40% of total Fc/2) corresponding to G0F with 2×GalNAz and two minor products (observed mass 25636 Da, approximately 30% of total Fc/2) for G1 F with 1×GalNAz and (observed mass 25474 Da, approximately 20% of total Fc/2) for G0F with 1×GalNAz. Approximately 10% of G1F starting material was also observed (25392 Da).

### Example 2. Galactose and Neuraminic Acid Trimming of Trastuzumab-(M5G1FS1)₂ to Trastuzumab-(M5G0F)

Trastuzumab expressed in the presence of swainsonine (as in FIG. 8C) (10.5 mg, 15 mg/mL) was incubated with neuraminidase (0.5 mU/mg IgG) from Vibrio cholerae (commercially available from Sigma-Aldrich) and β(1,4)-galactosidase (0.9 mU/mg IgG) from Streptococcus pneumoniae (commercially available from QA-Bio) in 50 mM sodium acetate pH 6.0 and 5 mM CaCl₂ at 37° C. for 16 hrs. A single major heavy chain product was observed corresponding to trastuzumab-(M5G0F) (50712 Da, >90% of total heavy chain product). Subsequently the solution was dialyzed to 20 mM tricine buffer pH 8.0 (3×) and concentrated using a Vivaspin Turbo 4 ultrafiltration unit (Sartorius).

### Example 3. Enzymatic Remodeling of Trastuzumab M5G0F to Trastuzumab-(M5G0F-6-5 azidoGalNAc)₂

Trastuzumab-(M5G0F)₂ (5.5 mg, 20 mg/mL) was incubated with TnGalNAcT (12.5% w/w), UDP 6-azidoGalNAc (75 eq compared to IgG), as described in WO2016170186, incorporated by reference, and calf intestine alkaline phosphatase (0.01% w/w, Roche) in 10 mM MnCl₂ and tricine buffer pH 8.0 for 16 hours at 30° C. The functionalized IgG was purified using a protA column (25 mL, CaptivA PriMAB). After loading of the reaction mixture, the column was washed with TBS+0.2% triton and TBS. The IgG was eluted with 0.1 M NaOAc pH 2.7 and neutralized with 2.5 M Tris-HCl pH 7.2. After three times dialysis to PBS, the functionalized trastuzumab was concentrated using a Vivaspin Turbo 4 ultrafiltration unit (Sartorius). Mass spectral analysis of a sample after IdeS treatment showed one major Fc/2 product (observed mass 25580 Da, approximately 90% of total Fc/2) corresponding to M5G0F with 1×6-azido GalNAc.

### Example 4. Galactose Trimming of Trastuzumab to Trastuzumab-(G0F)₂

Trastuzumab (11.5 mg, 20 mg/mL) was incubated with β(1,4)-galactosidase (60 mU/mg IgG) from Streptococcus pneumoniae (commercially available from QA-Bio) in 50 mM sodium phosphate buffer pH 6.0 at 37° C. After 16 hrs, additional β(1,4)-galactosidase (30 mU/mg IgG) was added and incubated again at 37° C. for 16 hrs. A single major heavy chain product was observed corresponding to trastuzumab-(G0F)₂ (25232 Da). Subsequently the solution was dialyzed to 100 mM histidine buffer pH 6.5 (3×) and concentrated using a Vivaspin Turbo 4 ultrafiltration unit (Sartorius).

### Example 4b. Galactose Trimming of Trastuzumab to Trastuzumab-(G0F)₂

Trastuzumab (55 mg, 20 mg/mL) was incubated with β(1,4)-galactosidase (2 mU/mg IgG) from Streptococcus pneumoniae (commercially available from QA-Bio) in 50 mM sodium phosphate buffer pH 6.0 at 37° C. A single major heavy chain product was observed corresponding to trastuzumab-(G0F)₂ (25232 Da). Subsequently the solution was buffer exchanged using a HiTrap 26-10 desalting column (Cytiva), rinsed with 0.1M NaOH and equilibrated with 100 mM histidine, 150mM NaCl buffer pH 6.5, and concentrated using a Vivaspin Turbo 4 10 kDa MWCO ultrafiltration unit (Sartorius).

### Example 5. Enzymatic Remodeling Towards Bisected Trastuzumab-(G0FB-GlcNAz)₂

Trastuzumab-(G0F)₂ (2 mg, 8 mg/mL) was incubated with MGAT-3 (4% w/w, commercially available from R&D systems), UDP GlcNAz (50 eq compared to IgG) and calf intestine alkaline phosphatase (0.01% w/w, Roche) in 5 mM MnCl₂ and 100 mM histidine buffer pH 6.5 for 16 hours at 37° C. The functionalized IgG was dialyzed to PBS using Amicon Ultra spinfilter 0.5 mL MWCO kDa (Merck Millipore). Mass spectral analysis of a sample after IdeS treatment showed one major Fc/2 product (observed mass 25476 Da) corresponding to G0FB with 1×GlcNAz.

### Example 5b. Enzymatic Remodeling Towards Bisected Trastuzumab-(G0FB-GlcNAz)₂

Trastuzumab-(G0F)₂ (50 mg, 15 mg/mL) was incubated with MGAT-3 (1.5% w/w, commercially available from R&D systems), UDP GlcNAz (50 eq compared to IgG) and calf intestine alkaline phosphatase (0.01% w/w, Roche) in 10 mM MnCl₂ and 100 mM histidine buffer pH 6.5 for 16 hours at 37° C. The functionalized IgG was purified using a protA column (5 mL, MabSelect™ Sure™, Cytiva, as described in example 1). Subsequently the solution was buffer exchanged using a HiTrap 26-10 desalting column (Cytiva), rinsed with 0.1M NaOH and equilibrated with TBS pH 7.5. The IgG was concentrated using Vivaspin Turbo 4 10 kDa MWCO ultrafiltration unit (Sartorius). Mass spectral analysis of a sample after IdeS treatment showed one major Fc/2 product (observed mass 25476 Da) corresponding to G0FB with 1×GlcNAz.

### Example 6. Mannose Trimming of Trastuzumab-(M9)₂

Trastuzumab-(M9)₂ expressed in the presence of kifunensin (see FIG. 8A) (19 mg, 5 mg/mL) was incubated with α-mannosidase (2.5% w/w) from Canavalia ensiformis (commercially available from Sigma-Aldrich) in 5 mM ZnSO₄ and 100 mM sodium acetate buffer pH 4.5 at 37° C. for 16 hrs. After IdeS digestion a distribution of Fc/2 peaks was observed corresponding to M3 (24678, 15%), M4 (24841 Da, 41%), M5 (25004 Da, 32%) and M6 (25164 Da, 12%). Subsequently the solution was dialyzed to 100 mM histidine buffer pH 6.5 (3×) and concentrated using a Vivaspin Turbo 4 ultrafiltration unit (Sartorius).

### Example 7. Enzymatic Remodeling of Trastuzumab-(M5)₂ to Trastuzumab-(M5-GlcNAz)₂

Trastuzumab-(M5)₂ (2 mg, 8 mg/mL) was incubated with MGAT-1 (5% w/w), UDP GlcNAz (50 eq compared to IgG) and calf intestine alkaline phosphatase (0.01% w/w, Roche) in 10 mM MnCl₂ and 100 mM histidine buffer pH 6.5 for 16 hours at 37° C. The functionalized IgG was purified using a protA column (25 mL, CaptivA PriMAB). After loading of the reaction mixture, the column was washed with TBS+0.2% triton and TBS. The IgG was eluted with 0.1 M NaOAc pH 2.7 and neutralized with 2.5 M Tris-HCl pH 7.2. After three times dialysis to PBS, the functionalized trastuzumab was concentrated using a Vivaspin Turbo 4 ultrafiltration unit (Sartorius). Mass spectral analysis of a sample after IdeS treatment showed a distribution of peaks corresponding to M4-GlcNAz (25087 Da, approximately 15%), M5-GlcNAz) (25247 Da, approximately 30%), M6G0GlcNAz (25408 Da, approximately 15%).

### Example 8. Galactose Remodeling to Trastuzumab-(G2F)₂

Trastuzumab (5 mg, 20 mg/mL) was incubated with β(1,4)-GalT (3% w/w), calf intestine alkaline phosphatase (0.01% w/w, Roche) and UDP galactose (20 equivalents compared to IgG) in 20 mM MnCl₂ and 50 mM MOPS buffer pH 7.2 at 37° C. After 16 hrs, additional β(1,4)-GalT (1.5% w/w) and UDP galactose (10 equivalents compared to IgG) were added and incubated again at 37° C. for 16 hrs. The functionalized IgG was purified using a protA column (25 mL, CaptivA PriMAB). After loading of the reaction mixture, the column was washed with TBS+0.2% triton and TBS. The IgG was eluted with 0.1 M NaOAc pH 2.7 and neutralized with 2.5 M Tris-HCl pH 7.2. After three times dialysis to PBS the functionalized trastuzumab was concentrated using a Vivaspin Turbo 4 ultrafiltration unit (Sartorius). A single major heavy chain product was observed corresponding to trastuzumab-G2F (25555 Da). Subsequently the solution was dialyzed to 50 mM cacodylate buffer pH 7.2 (3×) and concentrated using a Vivaspin Turbo 4 ultrafiltration unit (Sartorius).

### Example 8b. Galactose Remodeling to Trastuzumab-(G2F)₂

Trastuzumab (50 mg, 15 mg/mL) was incubated with β(1,4)-GalT (Y289F) (1.25% w/w), calf intestine alkaline phosphatase (0.01% w/w, Roche) and UDP galactose (30 equivalents compared to IgG) in 6 mM MnCl₂ and TBS buffer pH 7.5 at 37° C. The functionalized IgG was buffer exchanged to 50 mM cacodylate buffer pH 7.2 using a HiTrap 26-10 desalting column (Cytiva), rinsed with 0.1M NaOH and equilibrated with and the functionalized trastuzumab was concentrated using a Vivaspin Turbo 4 10 kDa MWCO ultrafiltration unit (Sartorius). A single major heavy chain product was observed corresponding to trastuzumab-G2F (25555 Da).

### Example 9. Enzymatic Remodeling of Trastuzumab-(G2F)₂ to Trastuzumab-(G2F-9-N₃-Neu5Ac)₂

Trastuzumab-(G2F)₂ (0.2 mg, 3 mg/mL) was incubated with rhST6Gal1 (5% w/w, commercially available from R&D systems), CMP 9-N₃-Neu5Ac (20 eq compared to IgG) and calf intestine alkaline phosphatase (0.01% w/w, Roche) in 10 mM MnCl₂, 5 mM CaCl₂ and 50 mM cacodylate buffer pH 7.6 for 16 hours at 37° C. The functionalized IgG was dialyzed to PBS using Amicon Ultra spinfilter 0.5 mL MWCO 10 kDa (Merck Millipore). Mass spectral analysis of a sample after IdeS treatment showed one major Fc/2 product (observed mass 25871 Da) corresponding to G2F with 1×9-N₃-Neu5Ac and one minor Fc/2 product corresponding to G2F with 2×9-N₃-Neu5Ac.

### Example 9b. Enzymatic Remodeling of Trastuzumab-(G2F)₂ to Trastuzumab-(G2F-N₃-Neu5Ac)₂

Trastuzumab-(G2F)₂ (40 mg, 10 mg/mL) was incubated with ST6Gal1 (1% w/w), CMP-Neu5AcN₃ (10 eq compared to IgG) and calf intestine alkaline phosphatase (0.01% w/w, Roche) in 6 mM MnCl₂, 50 mM cacodylate buffer pH 7.6 for 16 hours at 37° C. The functionalized IgG was purified using a protA column (5 mL, MabSelect™ Sure™, Cytiva, as described in example 1). Subsequently the solution was dialyzed to TBS and concentrated using Vivaspin Turbo 4 ultrafiltration units (Sartorius). Mass spectral analysis of a sample after IdeS treatment showed one major Fc/2 product (observed mass 25885 Da) corresponding to G2F with 1×Neu5AcN₃ and one minor Fc/2 product (observed mass 26218 Da) corresponding to G2F with 2×Neu5AcN_{3.}

### Preparation of ADCs

Antibody-drug-conjugates by conjugation of compound 3a to the remodeled antibodies.

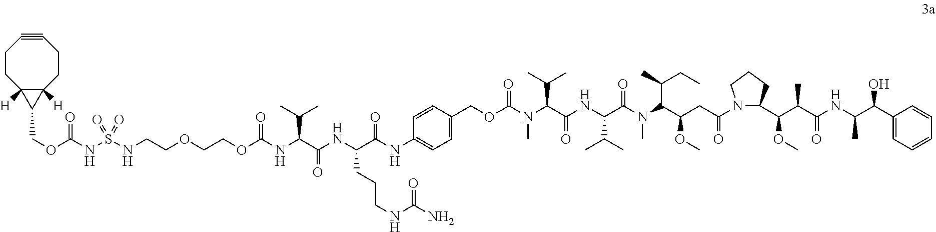

### Example 10. Trastuzumab-(GIF-6-azidoGalNAc-3a)₂ (SiteClick™+6-N₃-GalNAc+MMAE)

To a solution of trastuzumab-(G1F-6-azidoGalNAc)₂ (112 μL, 3 mg, 20 mg/ml in PBS pH 7.4) was added sodium deoxycholate (110 mM, 15 μL) and compound 3a (15 μL, 20 mM solution in DMF, 15 eq compared to IgG) followed by overnight incubation at rt. The ADC was diluted in PBS and purified on a Superdex200 Increase 10/300 GL (GE Healthcare) on an AKTA Purifier-10 (GE Healthcare). Mass spectral analysis of the IdeS-digested sample showed two major products, corresponding to the conjugated Fc/2 fragment with 2×compound 3a (observed mass 28709 Da, approximately 30% of total Fc/2 fragment), and the Fc/2 fragment with 1×compound 3a (observed mass 27132 Da, approximately 70% of total Fc/2 fragment). The calculated drug:antibody ratio (DAR), determined using RP-HPLC, was 1.63.

### Example 11. Trastuzumab-(6-azidoGalNAc-3a)₂ (GlycoConnect™+6-N₃-GalNAc+MMAE)

To a solution of trastuzumab-(6-azidoGalNAc)₂ (136 μL, 4.5 mg, 15 mg/ml in PBS pH 7.4), prepared according to WO2016170186, in PBS (134 μL) was added compound 3a (30 μL, 10 mM solution in DMF, 10 eq compared to IgG) followed by overnight incubation at rt. The ADC was diluted in PBS and purified on a Superdex200 Increase 10/300 GL (GE Healthcare) on an AKTA Purifier-10 (GE Healthcare). Mass spectral analysis of the IdeS-digested sample showed one major product, corresponding to the conjugated Fc/2 fragment with compound 3a (observed mass 25874 Da, approximately 75% of total Fc/2 fragment) and a minor peak corresponding to fragmentation of the vc-PABC linker (25114 Da, approximately 25% of total Fc/2 fragment). The calculated DAR was 1.84.

### Example 12. Trastuzumab-(M5F-6-azidoGalNAc-3a)₂

To a solution of trastuzumab-(M5F-6-azidoGalNAc)₂ (81 μL, 2.8 mg, 15 mg/ml in PBS pH 7.4) was added compound 3a (46 μL, 1 mM solution in DMF, 10 eq compared to IgG) followed by overnight incubation at rt. The ADC was diluted in PBS and purified on a Superdex200 Increase 10/300 GL (GE Healthcare) on an AKTA Purifier-10 (GE Healthcare). Mass spectral analysis of the IdeS-digested sample showed one major product, corresponding to the conjugated Fc/2 fragment with compound 3a (observed mass 27091 Da, approximately 65% of total Fc/2 fragment) and a minor peak corresponding to fragmentation of the vc-PABC linker (26330 Da, approximately 35% of total Fc/2 fragment). The calculated DAR was 1.75.

### Example 13. Trastuzumab-(G0FB-GlcNAz-3a)₂

To a solution of trastuzumab-(G0FB-GlcNAz)₂ (78 μL, 1.9 mg, 15 mg/ml in PBS pH 7.4) was added sodium deoxycholate (110 mM, 12.5 μL) and compound 3a (12.5 μL, 15 mM solution in DMF, 15 eq compared to IgG) followed by overnight incubation at rt. The ADC was diluted in PBS and purified on a Superdex200 Increase 10/300 GL (GE Healthcare) on an AKTA Purifier-10 (GE Healthcare). Mass spectral analysis of the IdeS-digested sample showed one major product, corresponding to the conjugated Fc/2 fragment with 1×compound 3a (observed mass 26986 Da). The calculated DAR was 1.68.

### Example 14. Trastuzumab-(M5-GlcNAz-3a)₂

To a solution of trastuzumab-(M5-GlcNAz)₂ (490 μL, 9.8 mg, 15 mg/ml in PBS pH 7.4) was added sodium deoxycholate (110 mM, 65 μL) and compound 3a (65 μL, 10 mM solution in DMF, 10 eq compared to IgG) followed by overnight incubation at rt. The ADC was diluted in PBS and purified on a Superdex200 Increase 10/300 GL (GE Healthcare) on an AKTA Purifier-10 (GE Healthcare). The product was then purified over a HIC, 4.6 mL Hi Screen butyl HP column. A gradient from 1M (NH₄)₂SO₄ in 50 mM phosphate pH 7.0 to 10% MeCN in 50 mM phosphate pH 7.0 was used. Lastly the ADC was dialyzed to PBS. Mass spectral analysis of the DAR2-IdeS-digested sample showed mainly MSGO(MMAE) (26758 Da). The calculated DAR was 1.84.

### Example 15. Trastuzumab-(G2F-9-azido-Neu5Ac-3a)₂

To a solution of trastuzumab-(G2F-9-azido-Neu5Ac)₂ (55 μL, 0.2 mg, 3.5 mg/ml in PBS pH 7.4) was added sodium deoxycholate (110 mM, 5.5 μL) and compound 3a (5.5 μL, 2.3 mM solution in DMF, 10 eq compared to IgG) followed by overnight incubation at rt. The ADC was dialyzed to PBS using Amicon Ultra spin-filter 0.5 mL MWCO 10 kDa (Merck Millipore). Mass spectral analysis of the IdeS-digested sample showed one major product, corresponding to the conjugated Fc/2 fragment with 1×compound 3a (observed mass 27381 Da). The calculated DAR was 1.74.

### Example 16. Expression of Trastuzumab in Presence of Swainsonine

Trastuzumab was transiently expressed in CHO K1 cells by Evitria (Zurich, Switzerland) in the presence of 25 μg/mL swainsonine (commercially available from Sigma-Aldrich), purified using protein A sepharose and analyzed by mass spectrometry. Both concentrations of swainsonine gave three major heavy chain products of trastzumab which correspond to the trastuzumab heavy chain substituted with MSG0F (50716 Da, ±24% of total heavy chain product), M5G1F (50878 Da, ±43% of total heavy chain product), and M5G1FS1 (51169 Da, ±33% of total heavy chain product).

### Example 17. Expression of Trastuzumab in Presence of Kifunensin

Trastuzumab was transiently expressed in CHO K1 cells by Evitria (Zurich, Switzerland) in the presence of kifunensin (commercially available from Sigma-Aldrich), purified using protein A sepharose and analyzed by mass spectrometry. One major peak corresponding to the Fc/2 fragment of trastzumab-M9 was detected (25654 Da, ±93% of total heavy chain product).

### Example 18. Expression and Isolation of GlcNAc-T1 (GnT-I)

The sequence coding for amino acids 31 to 416 of human mannosyl (α-1,3-)-glycoprotein α-(1,2)-N-acetylglucosaminyltransferase (N-acetylglucosaminyltransferase I, GnT-I) was PCR amplified from human placenta cDNA using the primers 5′-agctCATATGcgcccagcacctgg and 5′-agctGGATCCctaattccagctaggatcatagccctc and cloned into the NDel and BamHI sites of pET16B. GnT-I was expressed and isolated according to the reported procedure by Tolbert et al. Advanced Synthesis& Catalysis 2008, 350, 1689-1695, incorporated by reference.

### Example 19. E. coli expression of His₆-GlcNAc-T1 and Inclusion Body Isolation

Expression of His₆-GlcNAc-T1 starts with the transformation of the plasmid (pET16B-GnT1) into BL21 cells (Novagen). Next step was the inoculation of 500 mL culture (LB medium+ampicillin) with BL21 cells. When OD600 reached 1.5, cultures were induced with 1 mM IPTG (500 μL of 1M stock solution). After >16 hours induction at 16° C., the culture was pelleted by centrifugation. The cell pellet gained from 500 mL culture was lysed in 25 mL BugBuster™ with 625 units of benzonase and incubated on roller bank for 30 min at room temperature. After lysis the insoluble fraction was separated from the soluble fraction by centrifugation (15 minutes, 15000×g). The insoluble fraction was dissolved in 25 mL BugBuster™ with lysozyme (final concentration: 200 μg/mL) and incubated on the roller bank for 10 min. Next the solution was diluted with 6 volumes of 1:10 diluted BugBuster™ and centrifuged 15 min, 15000×g. The pellet was resuspended in 250 mL of 1:10 diluted BugBuster™ by using the homogenizer and centrifuged at 15 min, 12000×g. The last step was repeated 3 times.

### Example 20. Refolding of His₆-GlcNAc-T1 from Isolated Inclusion Bodies

The purified inclusion bodies containing His₆-GlcNAc-T1 (MGAT-1), were dissolved and denatured in 30 mL 5 M guanidine with 40 mM Cysteamine and 20 mM Tris pH 8.0. The suspension was centrifuged at 16.000×g for 5 min to pellet the remaining cell debris. The supernatant was diluted to 1 mg/mL with 5 M guanidine with 40 mM Cysteamine and 20 mM Tris pH 8.0 and incubated for 2 hours at RT on a roller-bank. The 1 mg/mL solution is added dropwise to 10 volumes of refolding buffer (50 mM Tris, 10.53 mM NaCl, 0.44 mM KCl, 2.2 mM MgCl₂, 2.2 mM CaCl₂, 0.055% PEG-4000, 0.55 M L-arginine, 4 mM cysteamine, 4 mM cystamine, at pH 8.0) in a cold room at 4° C., stirring required. The solution was left at 4° C. for 72 h. The solution was dialyzed to 10 mM NaCl and 20 mM Tris pH 8.0, 1× overnight and 2×4 hours, using a Spectrum™ Spectra/Por™ 3 RC Dialysis Membrane Tubing 3500 Dalton MWCO. Refolded His₆-GlcNAc-T1 was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 mM NaCl, pH 8.0) on a gradient of 30 mL from buffer A to buffer B. Fractions were analysed by SDS-PAGE on polyacrylamide gels (12%). Mass spectral analysis showed a weight of 49322 Da (expected: 49329 Da). The product was stored at −80° C. prior to further use.

### Example 21. Binding Affinity of ADCs with CD64 and CD16A Using Biacore

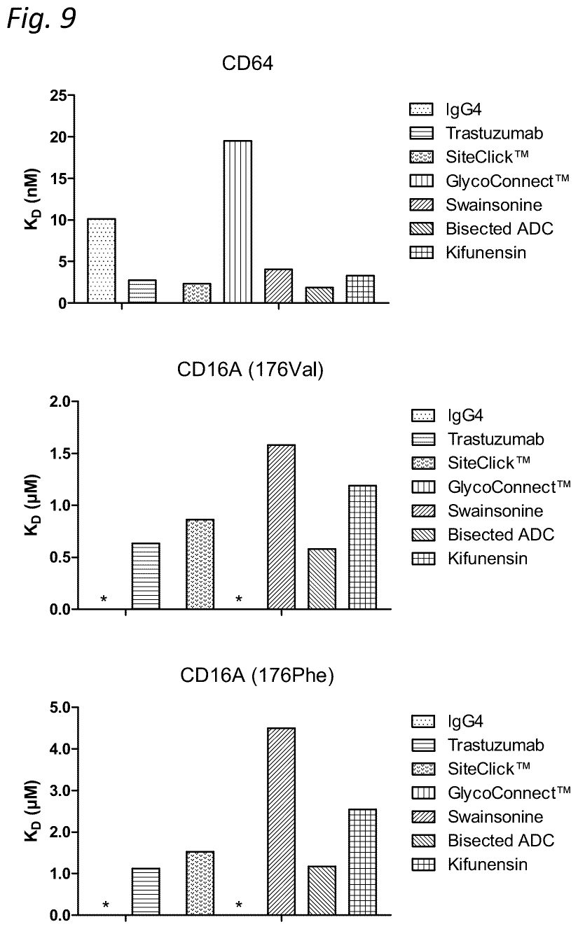

His₆-tagged Fc gamma receptors are captured on a CM5 chip previously coupled with an anti-HIS antibody (9000 RU) by standard amine coupling. Increasing concentrations of antibody-drug conjugate (five point three-fold dilution in HBS-P+ buffer) are subsequently injected over the antigen (either CD64 or CD16A, loaded to ˜30 RU at 10 μl/min) and a single dissociation is performed (single cycle kinetics). For the high affinity receptor FcγRI (CD64), 1:1 kinetic analysis is applied to investigate binding. Association time used is 200 s and dissociation time is 300 s. For the low affinity FcγRIIIA (CD16A Val and Phe) receptor steady state affinity is measured to investigate binding. Association time used is 30 s and dissociation time is 25 s. The instrument used is a Biacore T200, running Biacore T200 Evaluation Software V 2.0.1. Running buffer used is HBS-P+buffer at a flow rate of 30 μl/min. Regeneration is performed using two injections glycine pH 1.5. Results are depicted in FIG. 9 and in the Table below.

TABLE 1
Binding of different ADCs to FcγRI (CD64) and FcγRIIIA (CD16A,
176Val and 176Phe mutant) as determined by Biacore.

| | | FcγRIIIA | FcγRIIIA |
|---|---|---|---|
| | FcγRI | (176 Val) | (176 Phe) |
| IgG4 | 1.01 · 10⁻⁸ | — | — |
| Trastuzumab | 2.75 · 10⁻⁹ | 6.34 · 10⁻⁷ | 1.12 · 10⁻⁶ |
| SiteClick ™ | 2.33 · 10⁻⁹ | 8.64 · 10⁻⁷ | 1.53 · 10⁻⁶ |
| GlycoConnect ™ | 1.95 · 10⁻⁸ | — | — |
| Swainsonine | 4.07 · 10⁻⁹ | 1.58 · 10⁻⁶ | 4.50 · 10⁻⁶ |
| Bisected | 1.88 · 10⁻⁹ | 5.81 · 10⁻⁷ | 1.17 · 10⁻⁶ |
| Kifunensin | 3.29 · 10⁻⁹ | 1.19 · 10⁻⁶ | 2.54 · 10⁻⁶ |

Legend: SiteClick™=ADC based on conjugation of 3a (MMAE) to 6-N₃-GalNAc, attached to terminal GlcNAc in G0(F) glycoform; GlycoConnect™=ADC based on conjugation of 3a (MMAE) to 6-azidoGalNAc, attached to core GlcNAc (after trimming with endoglycosidase); Swainsonine=ADC based on conjugation of 3a (MMAE) to 6-N₃-GalNAc, attached to GlcNAc in M5(F) glycoform of antibody expressed in presence of inhibitor swainsonine (FIG. 8C); Bisected=ADC based on conjugation of 3a (MMAE) to GlcNAz, attached to mannose M1 in G0(F) glycoform; Kifunensin =ADC based on conjugation of 3a (MMAE) to GlcNAz, attached to mannose on M5 glycoform of antibody expressed in presence of inhibitor kifunensin (FIG. 8A).

Antibody-drug-conjugates by conjugation of compound 5a to the remodeled antibodies.

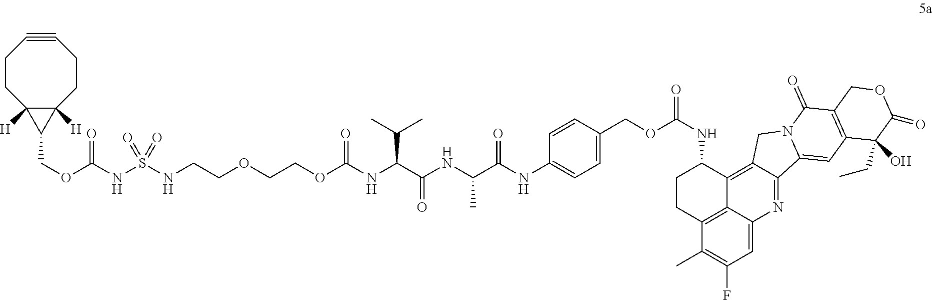 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)OCCOCCNS(=O)(=O)NC(=O)OC[C@@H]2[C@@H]4CCC#CCC[C@@H]42)C(C)C)cc1)CC3]

### Example 22. Trastuzumab-(G1F-GalNAz-5a)₂ (SiteClick™+GalNAz+exatecan)

To a solution of trastuzumab-(G1F-GalNAz)₂ (38 mg, 10 mg/ml in TBS pH 7.5) was added sodium deoxycholate (110 mM, 377 μL) and compound 5a (50 μL, 10 mM solution in DMF, 2 eq compared to IgG) in 30% (1081 μL) PG followed by overnight incubation at rt. After 16 h another 0.5 eq compound 5a (12.5 μL, 10 mM solution in DMF) was added in 5% PG (176 μL) for 2 h. The ADC was diluted in PBS and purified on a Superdex200 Increase 16/600 GL (GE Healthcare) on an AKTA Pure (GE Healthcare). The functionalized IgG was buffer exchanged to 20 mM histidine, 6% sucrose pH 6.0 using a HiTrap 26-10 desalting column (Cytiva). 0.04% Tween-20 was added before filter sterilization. Mass spectral analysis of the IdeS-digested sample showed two major products, corresponding to the conjugated Fc/2 fragment with 2×compound 5a (observed mass 27999 Da, approximately 60% of total Fc/2 fragment), and the Fc/2 fragment with 1×compound 5a (observed mass 26777 Da, approximately 40% of total Fc/2 fragment). The calculated drug:antibody ratio (DAR), determined using RP-HPLC, was 1.65.

### Example 23. Trastuzumab-(6-azidoGalNAc-5a)₂ (GlycoConnect™+6-N₃-GalNAz+exatecan)

To a solution of trastuzumab-(6-azidoGalNAc)₂ (38 mg, 10 mg/ml in TBS pH 7.5), prepared according to WO2016170186, was added compound 5a (50 μL, 10 mM solution in DMF, 2 eq compared to IgG) in 30% (1088 μL) PG followed by overnight incubation at rt. After 16 h another 0.25 eq compound 5a (6.3 μL, 10 mM solution in DMF) was added in 5% PG (183 μL) for 2 h.The ADC was diluted in PBS and purified on a Superdex200 Increase 16/600 GL (GE Healthcare) on an AKTA Pure (GE Healthcare). The functionalized IgG was buffer exchanged to 20 mM histidine, 6% sucrose pH 6.0 using a HiTrap 26-10 desalting column (Cytiva). 0.04% Tween-20 was added before filter sterilization. Mass spectral analysis of the IdeS-digested sample showed one major product, corresponding to the conjugated Fc/2 fragment with compound 5a (observed mass 25502 Da). The calculated DAR was 1.63.

### Example 24. Trastuzumab-(G0FB-GlcNAz-5a)₂ (Bisected-exatecan)

To a solution of trastuzumab-(G0FB-GlcNAz)₂ (40 mg, 10 mg/ml in TBS pH 7.5) was added sodium deoxycholate (110 mM, 400 μL) and compound 5a (373 μL, 10 mM solution in DMF, 14 eq compared to IgG) in 30% (826 μL) PG followed by overnight incubation at rt. The ADC was diluted in PBS and purified on a Superdex200 Increase 16/600 GL (GE Healthcare) on an AKTA Pure (GE Healthcare). The functionalized IgG was buffer exchanged to 20 mM histidine, 6% sucrose pH 6.0 using a HiTrap 26-10 desalting column (Cytiva). 0.04% Tween-20 was added before filter sterilization. Mass spectral analysis of the IdeS-digested sample showed one major product, corresponding to the conjugated Fc/2 fragment with 1×compound 5a (observed mass 26614 Da). The calculated DAR was 1.65.

### Example 25. Trastuzumab-(G2F-Neu5AcN₃-5a)₂ (Sialic Acid-Exatecan)

To a solution of trastuzumab-(G2F-Neu5AcN₃)₂ (38 mg, 10 mg/ml in TBS pH 7.5) was added sodium deoxycholate (110 mM, 375 μL) and compound 5a (200 μL, 10 mM solution in DMF, 8 eq compared to IgG) in 30% (925 μL) PG followed by overnight incubation at rt. The ADC was diluted in PBS and purified on a Superdex200 Increase 16/600 GL (GE Healthcare) on an AKTA Pure (GE Healthcare). The functionalized IgG was buffer exchanged to 20 mM histidine, 6% sucrose pH 6.0 using a HiTrap 26-10 desalting column (Cytiva). 0.04% Tween-20 was added before filter sterilization. Mass spectral analysis of the IdeS-digested sample showed one major product, corresponding to the conjugated Fc/2 fragment with 1×compound 5a (observed mass 27027 Da). The calculated DAR was 1.83.

### Example 26. CD16a Binding Using ELISA

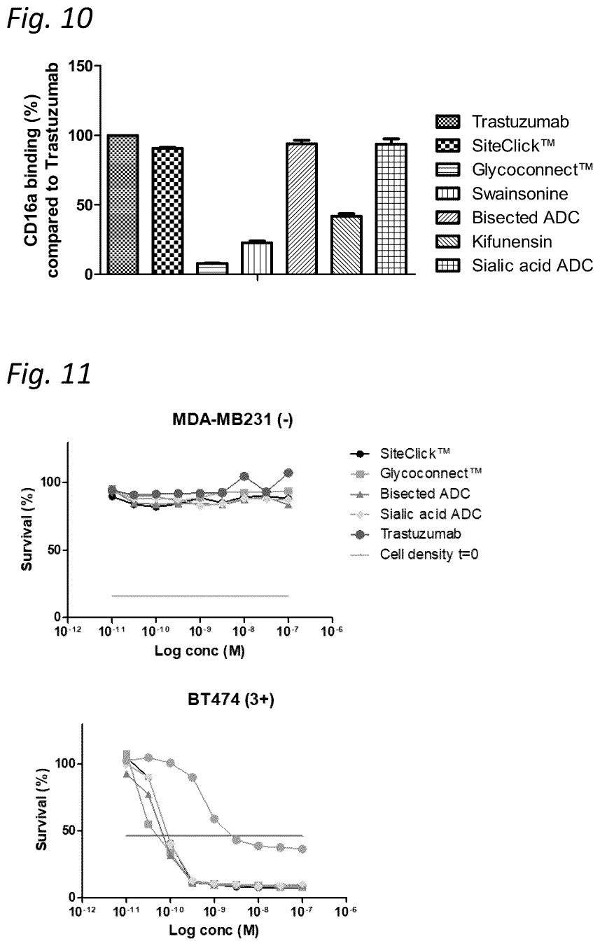

Nickel NTA plates (Pierce™ Nickel coated plated, ThermoScientific™) were washed three times prior to use. FcγRIIIA (CD16A, 176Val, His Tag, Sino Biological) was dissolved at a concentration of 2 μg/mL in 0.1% BSA in PBS (PBA). 100 μL was added to each well and incubated while shaking for 1 hour at room temperature. After removal, the plate was washed 3× with 0.05% Tween-20 in PBS (washing buffer). ADCs were diluted in 0.1% PBA to a final concentration of 8 pg/mL and 100 μL was added to each well (in quadruplo). ADCs were incubated for 1 h at room temperature. Prior to the addition of 100 μL 1:1000 dilution of secondary antibody (Goat anti-human IgG, HRP conjugate, Invitrogen) the plate was washed 3× with washing buffer. The plate was incubated again for 1 h at room temperature and subsequently washed 3× with washing buffer. Finally, 100 μL TMB ELISA substrate (1 Step™ Turbo TMB ELISA substrate, ThermoScientific™) was added and incubated for 15 minutes. To quench the reaction, 100 μL 2M H₂SO₄ was added and the absorbance of the colorimetric signal was measured with Infinite® M1000 (Tecan) at 450 nm. Data was plotted as percentage of trastuzumab (see FIG. 10)

### Example 27: In Vitro Cytotoxicity

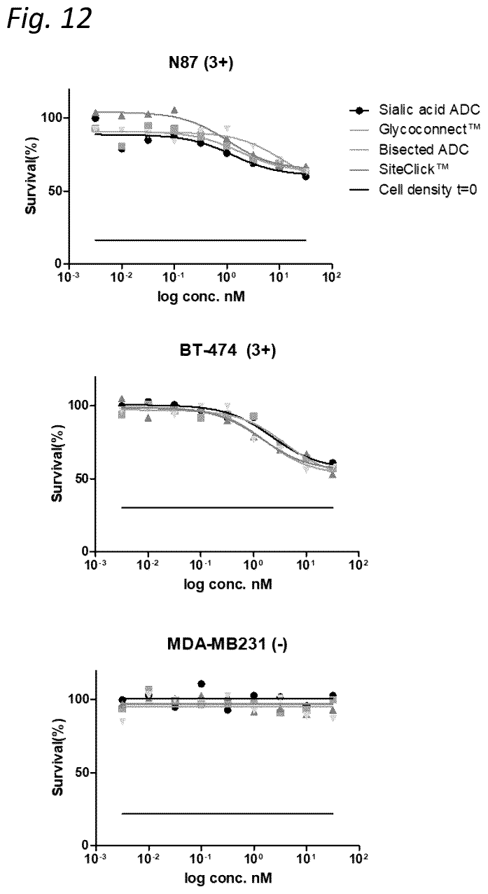

BT-474 (Her2 3+), N87 (Her2 3+) and MDA-MB231 (Her2−) cells were plated in 96-well plates (5000 cells/well) in RPMI 1640 GlutaMAX (Invitrogen) supplemented with 10% fetal bovine serum (FBS) (Invitrogen, 150 μL/well) and incubated overnight in a humidified atmosphere at 37° C. and 5% CO₂. ADCs were added in triplo in a square root of 10 dilution series to obtain a final concentration ranging from 5 pM to 30 nM. The cells were incubated for 5 days in a humidified atmosphere at 37° C. and 5% CO₂. The culture medium was replaced by 0.01 mg/mL resazurin (Sigma Aldrich) in RPMI 1640 GlutaMAX supplemented with 10% FBS (200 μL/well). After approximately 4 hours in a humidified atmosphere at 37° C. and 5% CO₂ the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability (see FIGS. 11 and 12). IC₅₀ values for ADCs on BT474 and N87 were calculated by non-linear regression using Graphpad prism software and are shown in the table below.

| | MMAE-ADCs | Exatecan-ADCs | Exatecan-ADCs |
|---|---|---|---|
| | on BT474 | on BT474 | on N87 |
| SiteClick ™ | 44.85 pM | 1.4 nM | 1.1 nM |
| Glycoconnect ™ | 11.58 pM | 4.0 nM | 1.8 nM |
| Bisected ADC | 36.22 pM | 1.7 nM | 11.6 nM |
| Sialic acid ADC | 47.55 pM | 2.2 nM | 1.3 nM |

### Example 28: In Vitro ADCC Assay

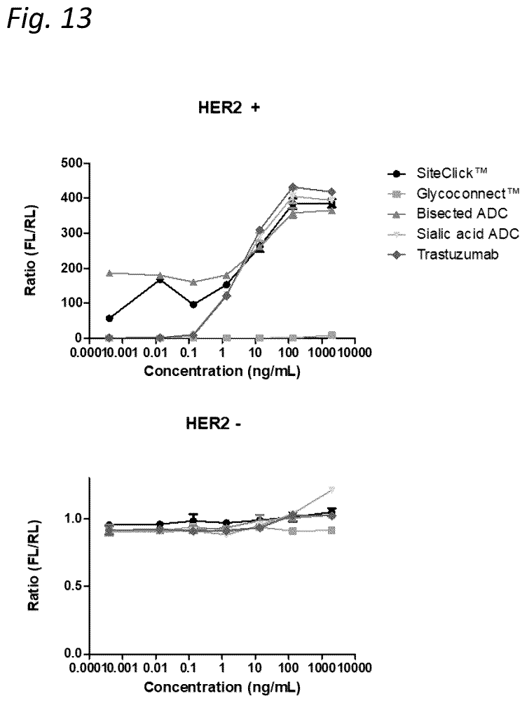

A serial dilution (8×) was made from ADCs in the range between 0-2000 ng/mL. 40 μL was added to each well, in duplo. iLite® ADCC effector FcγRIIIa (V), HER²(+) Target Assay ready cells and HER²(−) Target Assay ready cells (all from Svar Life Science) were thawed at 37° C. with gentle agitation. 250 μL ADCC effector cells was mixed with either HER²(+) or HER²(−) cells and diluted with 4.3 mL diluent (RPMI 1640+9% heat inactivated FBS+1% Penicillin Streptomycin). 40 μL diluted cells were added to test items and carefully mixed. The plates were incubated for 4 hours in a humidified atmosphere at 37° C. and 5% CO₂. In the meantime, substrate solutions were warmed to room temperature. Firefly luciferase substrate (Promega) was prepared using Dual Glow substrate and buffer solution and 80 μL was added per well. After 10 minute incubation at room temperature, luminescence was measured (using Envision multilabel plate reader). Next, Renilla luciferase substrate (Promega) is prepared by making a 1:100 dilution of dual Stop&Glo substrate with Stop&Go buffer. 80 μL was added to every well, and after 10 minute incubation, luminescence was measured again. The ratio between the readouts normalized the data for the number of cells (see FIG. 13).

### Example 29: Plasma Stability Test

Stability of ADCs in mice and human plasma was tested. Prior to the assay, the plasma was depleted from all IgG using ProtA purification (MabSelect™ Sure™, Cytiva) by collecting the flow through. ADCs were added to the depleted human/mouse serum to a final concentration of 0.1 mg/mL followed by incubation at 37° C. At each time point 0.5 mL was taken, snap frozen and stored at −80° C. until further analysis. CativA® Protein A Affinity Resin (Repligen) was washed 3× with PBS to remove storage EtOH. The resin was added to the samples and incubated 1 hour at room temperature. The resin was washed with PBS and subsequently 0.1 M Glycine-HCl pH 2.7 (0.4 mL) was added to elute the ADCs. After elution, the samples were immediately neutralized with 1.0 M Tris pH 8.0 (0.1 mL). The samples were spin filtrated using Amicon Ultra spin-filter 0.5 mL MWCO 10 kDa (Merck Millipore) to reduce the volume to 40 μL and a final concentration of approximately 1 mg/mL. Samples were analyzed on SE-HPLC to measure aggregation and RP-HPLC (DTT reduced) to determine the DAR, tables below.

Human plasma (MMAE-ADCs):

| | DAR | DAR | Monomer (%) | Monomer (%) |
|---|---|---|---|---|
| | T = 0 | T = 7 | T = 0 | T = 7 |
| Glycoconnect ™ | 1.82 | 1.82 | 91.1 | >98 |
| SiteClick ™ | 1.57 | 1.52 | 50.6 | 97.3 |
| Bisected ADC | 1.74 | 1.70 | 86.4 | 97.8 |
| Sialic Acid ADC | 1.49 | 1.47 | >98 | 94.8 |
| Trastuzumab | — | — | 97.6 | 98 |

Mice plasma (MMAE-ADCs):

| | DAR | DAR | Monomer (%) | Monomer (%) |
|---|---|---|---|---|
| | T = 0 | T = 7 | T = 0 | T = 7 |
| Glycoconnect ™ | 1.87 | 1.65 | 98.7 | 97.2 |
| SiteClick ™ | 1.42 | 0.78 | 97.3 | 96.7 |
| Bisected ADC | 1.73 | 1.34 | 96.7 | 94.2 |
| Sialic Acid ADC | 1.44 | 1.06 | 98.1 | 96.8 |
| Trastuzumab | — | — | 87.8 | 95.8 |

1. A method for binding to a cell comprising an Fc-gamma receptor, comprising contacting the cell with an antibody conjugate, wherein the antibody conjugate has structure (1):
Ab-[(GlcNAc(Fuc)_{b}-(G)ₑ-(Su-(Z-L-(D)ᵣ)ₓ)ₛ]_{y} (1)
wherein:
Ab is an antibody
GlcNAc is an N-acetylglucosamine moiety;
Fuc is a fucose moiety;
b is 0 or 1;
G is a monosaccharide;
e is an integer in the range of 4-10;
Su is a monosaccharide;
Z is a connecting group obtained by a cycloaddition or a nucleophilic reaction;
L is a linker;
D is a payload;
s is 1 or 2;
r is an integer in the range of 1-4;
x is 1 or 2;
y is 2 or 4.

2. The method according to claim 1, wherein the cell is an immune cell.

3. The method according to claim 2, wherein the immune cell is activated via binding to an Fc-gamma receptor expressed by the immune cell.

4. The method according to claim 3, wherein the Fc-gamma receptor is Fc-gamma receptor IA, IIA or IIIA.

5. The method according to claim 1, wherein the binding is improved over the binding of the same antibody conjugate but wherein e is below 4.

6. The method according to claim 1, wherein e=5, 6 or 7.

7. The method according to claim 1, wherein b=0.

8. The method according to claim 1, wherein G is selected from galactose, glucose, N-acetylgalactosamine, N-acetylglucosamine, mannose and N-acetylneuraminic acid.

9. The method according to claim 1, wherein (G)ₑ is according to structure (G1): 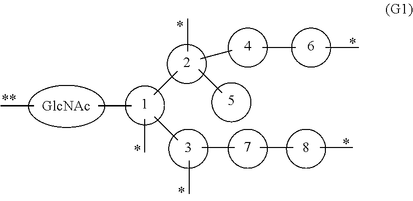
wherein:
(G)ₑ is connected to GlcNAc(Fuc)_{b} via the bond labelled with ** and to Su via one of the bonds labelled *;
monosaccharide (1) is Man;
monosaccharide (2) is Man or absent;
monosaccharide (3) is Man;
monosaccharide (4) is Man, GlcNAc or absent;
monosaccharide (5) is Man or absent;
monosaccharide (6) is Man, Gal or absent;
monosaccharide (7) is GlcNAc or absent;
monosaccharide (8) is Gal or absent.

10. The method according to claim 8, wherein:
(i) (1)=(2)=(3)=(4)=(5)=Man; (6)=(7)=(8)=absent; Su=GlcNAc and (G)ₑ is connected to Su via (3);
(ii) (1)=(2)=(3)=(4)=(5)=(6)=Man; (7)=(8)=absent; Su=GlcNAc and (G)ₑ is connected to Su via (3);
(iii) (1)=(2)=(3)=(4)=Man; (5)=(6)=(7)=(8)=absent; Su=GlcNAc and (G)ₑ is connected to Su via (3);
(iv) (1)=(2)=(3)=Man; (4)=(5)=(6)=(8)=absent; (7)=GlcNAc; Su=GalNAc and (G)ₑ is connected to Su via (7);
(v) (1)=(2)=(3)=(4)=Man; (5)=(6)=(8)=absent; (7)=GlcNAc; Su=GalNAc and (G)ₑ is connected to Su via (7);
(vi) (1)=(2)=(3)=(4)=(5)=Man; (6)=(8)=absent; (7)=GlcNAc; Su=GalNAc and (G)ₑ is connected to Su via (7);
(vii) (1)=(2)=(3)=Man; (4)=(7)=GlcNAc; (5)=(8)=absent; (6)=Gal; Su=GlcNAc and (G)ₑ is connected to Su via (1);
(viii) (1)=(2)=(3)=Man; (4)=(7)=GlcNAc; (5)=(6)=(8)=absent; Su=GlcNAc and (G)ₑ is connected to Su via (1);
(ix) (1)=(2)=(3)=Man; (4)=(7)=GlcNAc; (5)=(8)=absent; (6)=Gal; Su=GlcNAc and (G)ₑ is connected to Su via (3);
(x) (1)=(2)=(3)=Man; (4)=(7)=GlcNAc; (5)=(6)=(8)=absent; Su=GlcNAc and (G)ₑ is connected to Su via (3);
(xi) (1)=(2)=(3)=Man; (4)=(7)=GlcNAc; (5)=(8)=absent; (6)=Gal; Su=GalNAc and (G)ₑ is connected to Su via (7);
(xii) (1)=(2)=(3)=Man; (4)=(7)=GlcNAc; (5)=(6)=(8)=absent; Su=GalNAc and G)e is connected to Su via (7);
(xiii) (1)=(2)=(3)=Man; (4)=(7)=GlcNAc; (5)=(6)=absent; (8)=Gal; Su=Neu5Ac and (G)ₑ is connected to Su via (6) and (8);
(xiv) (1)=(2)=(3)=Man; (4)=(7)=GlcNAc; (5)=(8)=absent; (6)=Gal; Su=Neu5Ac and (G)ₑ is connected to Su via (6) and (6);
(xv) (1)=(2)=(3)=Man; (4)=(7)=GlcNAc; (5)=absent; (6)=(8)=Gal; Su=Neu5Ac and (G)ₑ is connected to Su via (6) and/or (8);
(xvi) (1)=(2)=(3)=Man; (4)=(5)=(6)=(7)=(8)=absent; Su=GlcNAc and (G)ₑ is connected to Su via (3);
(xvii) (1)=(3)=Man; (2)=(4)=(5)=(6)=(7)=(8)=absent; Su=GlcNAc and (G)ₑ is connected to Su via (3);
(xviii) (1)=(3)=Man; (2)=(4)=(5)=(6)=(8)=absent; (7)=GlcNAc; Su=GalNAc and (G)ₑ is connected to Su via (7).

11. The method according claim 9, wherein (G)ₑ is according to structure (G2):

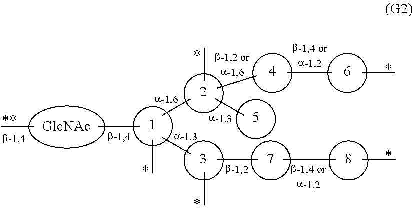

12. The method according to claim 1, wherein Su is selected from galactose, glucose, N-acetylgalactosamine, N-acetylglucosamine and N-acetylneuraminic acid.

13. The method according to claim 1, wherein the cycloaddition is a [4+2] cycloaddition or a 1,3-dipolar cycloaddition or the nucleophilic reaction is a Michael addition or a nucleophilic substitution; and/or wherein Z contains a triazole, a cyclohexene, a cyclohexadiene, a [2.2.2]-bicyclooctadiene, a [2.2.2]-bicyclooctene, an isoxazoline, an isoxazolidine, a pyrazoline, a piperazine, a thioether, an amide or an imide group.

14. The method according to claim 1, wherein one or more of the following applies: s=1; r=1 or 2; x=1; and y=2.

15. An antibody conjugate, wherein the antibody conjugate has structure (1):
Ab-[(GlcNAc(Fuc)_{b}-(G)ₑ-(Su-(Z-L-(D)ᵣ)ₓ)ₛ]_{y} (1)
wherein:
Ab is an antibody
GlcNAc is an N-acetylglucosamine moiety;
Fuc is a fucose moiety;
b is 0 or 1;
(G)ₑ is an oligosaccharide of structure (G1): 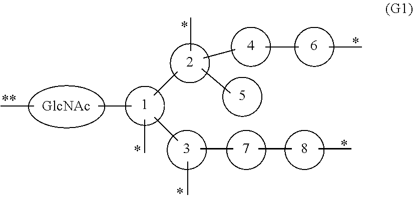
wherein (G)ₑ is connected to GlcNAc(Fuc)_{b} via the bond labelled with ** and to Su via one of the bonds labelled *; and
(i) (1)=(2)=(3)=(4)=(5)=Man; (6)=(7)=(8)=absent; and (G)ₑ is connected to Su via (3);
(ii) (1)=(2)=(3)=(4)=(5)=(6)=Man; (7)=(8)=absent; and (G)ₑ is connected to Su via (3);
(iii) (1)=(2)=(3)=(4)=Man; (5)=(6)=(7)=(8)=absent; and (G)ₑ is connected to Su via (3);
(vii) (1)=(2)=(3)=Man; (4)=(7)=GlcNAc; (5)=(8)=absent; (6)=Gal; and (G)ₑ is connected to Su via (1);
(viii) (1)=(2)=(3)=Man; (4)=(7)=GlcNAc; (5)=(6)=(8)=absent; and (G)ₑ is connected to Su via (1);
(ix) (1)=(2)=(3)=Man; (4)=(7)=GlcNAc; (5)=(8)=absent; (6)=Gal; and (G)ₑ is connected to Su via (3);
(x) (1)=(2)=(3)=Man; (4)=(7)=GlcNAc; (5)=(6)=(8)=absent; and (G)ₑ is connected to Su via (3);
e is an integer in the range of 4-10;
Su is a monosaccharide;
Z is a connecting group obtained by a cycloaddition or a nucleophilic reaction;
L is a linker;
D is a payload;
s is 1 or 2;
r is an integer in the range of 1-4;
x is 1 or 2;
y is 2 or4.

16. The antibody conjugate according claim 15, wherein (G)ₑ is according to structure (G2):

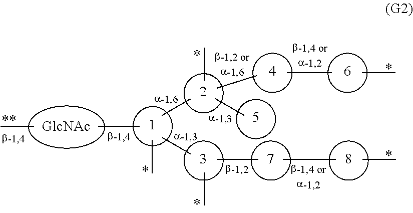

17. The antibody conjugate according claim 15, wherein e=5, 6 or 7.

18. The antibody conjugate according to claim 15, wherein b=0.

19. The antibody conjugate according to claim 15, wherein Su is selected from galactose, glucose, N-acetylgalactosamine, N-acetylglucosamine and N-acetylneuraminic acid.

20. The antibody conjugate according to claim 19, wherein Su is selected from N-acetylgalactosamine, N-acetylglucosamine or N-acetylneuraminic acid.

21. The antibody conjugate according to claim 15, wherein the cycloaddition is a [4+2] cycloaddition or a 1,3-dipolar cycloaddition or the nucleophilic reaction is a Michael addition or a nucleophilic substitution; and/or wherein Z contains a triazole, a cyclohexene, a cyclohexadiene, a [2.2.2]-bicyclooctadiene, a [2.2.2]-bicyclooctene, an isoxazoline, an isoxazolidine, a pyrazoline, a piperazine, a thioether, an amide or an imide group.

22. The antibody conjugate according to claim 15, wherein one or more of the following applies: s=1;r=1 or 2;x=1;and y=2.

23. A method for preparing an antibody conjugate according to claim 15, comprising:
(a) expressing an antibody in a mammalian expression system, optionally in the presence of a glycosidase or a glycosyltransferase inhibitor;
(b) optionally subjecting the expressed antibody to deglycosylation with an enzyme selected from an alpha-mannosidase, galactosidase and sialidase;
(c) contacting the optionally deglycosylated antibody with a saccharide moiety of structure Nuc-Su(F)ₓ in the presence of a glycosyltransferase to obtain a modified antibody having structure (2):
Ab-[(GlcNAc(Fuc)_{b}-(G)ₑ-(Su-(F)ₓ)ₛ]_{y} (2)
wherein
Ab is an antibody
GlcNAc is an N-acetylglucosamine moiety;
Fuc is a fucose moiety;
b is 0 or 1;
G is a monosaccharide;
e is an integer in the range of 4-10;
Su is a monosaccharide;
s is 1 or 2;
x is 1 or 2;
y is 2 or 4;
Nuc is a nucleotide; and
F is reactive moiety capable of reacting in a cycloaddition or a nucleophilic reaction;
(d) conjugating the modified antibody having structure (2) with a linker payload construct having structure (3):
Q-L-(D)ᵣ (3)
wherein L is a linker, D is a payload, and Q is reactive moiety capable of reacting with F in a cycloaddition or a nucleophilic reaction, to obtain an antibody conjugate having structure (1):
Ab-[(GlcNAc(Fuc)_{b}-(G)ₑ-(Su-(Z-L-(D)ᵣ)ₓ)ₛ]_{y} (1)
wherein Ab, GlcNAc, Fuc, G, Su, b, e, s, x, y, L, D are as defined above, r is an integer in the range of 1-4, and Z is a connecting group formed by the reaction of F with Q in a cycloaddition or a nucleophilic reaction.

24. The method according to claim 23, wherein the glycosidase inhibitor is a mannosidase inhibitor.

25. The method according to claim 24, wherein the mannosidase inhibitor is swainsonine or kifunensin.

26. The method according to claim 23, wherein the glycosyltransferase inhibitor is a fucosyltransferase inhibitor, a galactosyltransferase inhibitor or a sialyltransferase inhibitor.

27. The method according to claim 26, wherein the fucosyltransferase inhibitor is selected from the group of fucostatin I, fucostatin II, 2-fluorofucose, 6-fluorinated derivative of fucose, Fucotrim I and Fucotrim II, and acylated variants thereof.

28. A pharmaceutical composition comprising the antibody conjugate according to claim 14 and a pharmaceutically acceptable carrier.

29. A method of treating cancer, comprising administering to a patient in need thereof an antibody conjugate according to claim 15.
